(19) United States
(12) Patent Application Publication
(10) Pub. No.: US 2009/0274853 A1
(43) Pub. Date: Nov. 5, 2009

(54) DICHROIC DYE COMPOSITION

(75) Inventor: Shinichi Morishima, Minami-ashigara-shi (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Minato-ku (JP)

(21) Appl. No.: 12/410,822

(22) Filed: Mar. 25, 2009

(30) Foreign Application Priority Data
Mar. 31, 2008 (JP) 2008-092412

(51) Int. Cl. C09K 19/10; C07C 245/02

(52) U.S. Cl. 428/1.31; 534/577; 252/299.1

(57) ABSTRACT

A dichroic dye composition, containing at least one kind of azo dye represented by formula (I) that has a liquid crystallinity:

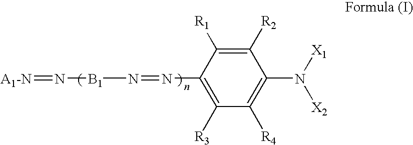

wherein R₁, R₂, R₃, R₄, X₁ and X₂ each independently represent a hydrogen atom or a substituent; A₁ is a substituted or unsubstituted phenyl group, a substituted or unsubstituted naphthyl group, or a substituted or unsubstituted aromatic heterocyclic group; B₁ is a divalent substituted or unsubstituted aromatic hydrocarbon group or a divalent substituted or unsubstituted aromatic heterocyclic group; n represents an integer of 1 to 5; and at least one of B₁s represents a phenylene group having an alkyl group.

### FIELD OF THE INVENTION

The present invention relates to a dichroic dye composition. Further, the present invention relates to a light absorption anisotropic film, a polarizing element and a liquid crystal display device, each employing the dichroic dye composition.

### BACKGROUND OF THE INVENTION

When a function such as an attenuation function, a polarization function, a scattering function and a light-shielding function is required to effect for an irradiated light including a laser light and a natural light employed, an apparatus which operates based on a different principle was adapted conventionally depending on the function required. Accordingly, products corresponding to the functions were prepared respectively by production processes that were different depending on the respective functions.

For example, in LCD (Liquid Crystal Display), linear polarizing plates or circular polarizing plates are used to control optical rotation or birefringence in display. Also in OLED (Organic Electroluminescence), circular polarizing plates are used to prevent reflection of external outside light. Heretofore, for such polarizing plates (polarizing elements), iodine has been widely used as a dichroic material. However, if iodine is used for a polarizing plate, its heat resistance or light fastness is inadequate since iodine is highly sublimable. Further, the extinction color becomes dark grayish blue, and an ideal achromatic color polarizing plate for the entire visible spectral region cannot necessarily be obtained.

Therefore, a polarizing element has been studied wherein an organic dye is used as a dichroic material which replaces iodine. However, such an organic dye has a problem such that only polarizing elements are obtainable which are distinctly inferior to those employing iodine for dichroic property. Particularly, a polarizing element is an important constituent in LCD employing as the display principle optical rotation or birefringence of light, and a new polarizing element has been developed for the purpose of improving display performance and the like in recent years.

As one method of forming such a polarizing element, a method may be mentioned wherein, in the same manner as in the case of a polarizing film containing iodine, an organic dye having dichroism (dichroic dye) is dissolved or adsorbed in a polymer material such as a polyvinyl alcohol, and the obtained film is stretched in one direction into a film so that the dichroic dye is oriented. However, this method had such a problem that time and effort are required for e.g. the stretching step.

Thus, other methods attract attention in recent years and as such methods, Dreyer, J. F., Journal de Physique, 1969, 4, 114., “light Polarization From Films of Lyotropic Nematic Liquid Crystals” discloses a method of orientating a dichroic dye on a substrate such as glass or a transparent film utilizing e.g. intermolecular interaction of organic dye molecules, to form a polarizing film (anisotropic dye film). However, it was known that there was a problem for heat resistant property in the method described in the above document.

Further, the method of orienting a dichroic dye on a substrate such as glass or a transparent film utilizing e.g. intermolecular interaction of organic dye molecules may be a wet system film-forming method. In a case where an anisotropic dye film is prepared by the wet system film-forming method, the dye molecules to be used for the dye film are required not only to show high degree of dichroism but also to be a dye suitable for the process for the wet system film-forming method. Examples of the process in the wet film-forming method include a process of disposing and orientating the dye on a substrate or a process of controlling the orientation. Therefore, there are many cases that even the conventional dyes that can be employed for the polarizing elements passing through the above-mentioned stretching treatment are not suitable for the wet film-forming method. Further, JP-A-2002-180052 (“JP-A” means unexamined published Japanese patent application), JP-A-2002-528758 and JP-A-2002-338838 propose materials suitable for the process of the wet system film-forming method. However, although such materials are suitable for the process, they have had such a drawback that they cannot show high dichroism.

Further, JP-T-8-511109 (“JP-T” means published searched patent publication) proposes a dye represented by chromogen (SO₃M)ₙ as a material suitable for the process. In the document, the achromatic color is given by combining several kinds of dichroic dyes each other. However, when an anisotropic dye film is obtained by combining the several kinds of dichroic dyes each other, a molecular orientation for mixing different molecules is disturbed and there was a problem that achieving a high dichroism is difficult.

### SUMMARY OF THE INVENTION

The present invention resides in a dichroic dye composition, comprising at least one kind of azo dye represented by formula (I) that has a liquid crystallinity:

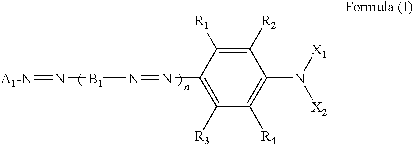

wherein R₁, R₂, R₃, R₄, X₁ and X₂ each independently represent a hydrogen atom or a substituent; A₁ is a substituted or unsubstituted phenyl group, a substituted or unsubstituted naphthyl group or a substituted or unsubstituted aromatic heterocyclic group; B₁ is a divalent substituted or unsubstituted aromatic hydrocarbon group or a divalent substituted or unsubstituted aromatic heterocyclic group; n represents an integer of 1 to 5; and at least one of B₁s represents a phenylene group having an alkyl group.

Further, the present invention resides in a light absorption anisotropic film formed by employing the above-described dichroic dye composition.

Further, the present invention resides in a polarizing element comprising an alignment film and the above-described light absorption anisotropic film on a support.

Further, the present invention resides in a liquid crystal display device comprising the above-described light absorption anisotropic film or the above-described polarizing element.

Furthermore, the present invention resides in a method of producing the above-described polarizing element, comprising the steps of:

(1) rubbing a support or an alignment film formed on a support;

(2) applying the above-described dichroic dye composition dissolved in an organic solvent on the rubbing treated support or alignment film; and

(3) orienting the dichroic dye composition by causing the organic solvent to evaporate.

Other and further features and advantages of the invention will appear more fully from the following description.

### DETAILED DESCRIPTION OF THE INVENTION

According to the present invention, there is provided the following means:

<1> A dichroic dye composition, comprising at least one kind of azo dye represented by formula (I) that has a liquid crystallinity:

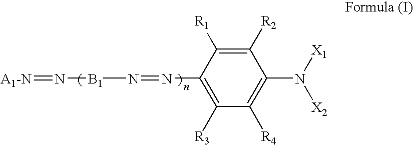

wherein R₁, R₂, R₃, R₄, X₁ and X₂ each independently represent a hydrogen atom or a substituent; A₁ is a substituted or unsubstituted phenyl group, a substituted or unsubstituted naphthyl group, or a substituted or unsubstituted aromatic heterocyclic group; B₁ is a divalent substituted or unsubstituted aromatic hydrocarbon group or a divalent substituted or unsubstituted aromatic heterocyclic group; n represents an integer of 1 to 5; and at least one of B₁s represents a phenylene group having an alkyl group.

<2> The dichroic dye composition described in the above item <1>, wherein, in formula (I), A₁ represents a substituted or unsubstituted phenyl group; B₁ represents a divalent substituted or unsubstituted phenylene group; and n represents an integer of 2 to 4.
<3> The dichroic dye composition described in the above item <1> or <2>, wherein the azo dye represented by formula (I) is a compound represented by formula (II):

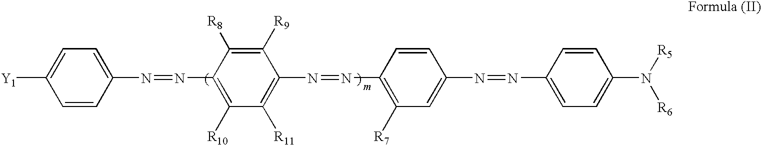

wherein R₅, R₆ and R₇ each independently represent an alkyl group; R₈, R₉, R₁₀ and R₁₁ each independently represent a hydrogen atom or a substituent; Y₁ represents a substituted or unsubstituted, alkyl group, alkenyl group, alkynyl group, aryl group, alkoxy group, alkoxycarbonyl group, acyloxy group, acylamino group, alkoxycarbonylamino group, sulfonylamino group, sulfamoyl group, carbamoyl group, alkylthio group, sulfonyl group or ureido group; and m represents an integer of 1 to 3.

<4> The dichroic dye composition described in any one of the above items <1> to <3>, further comprising at least one kind of fluoro-aliphatic group-containing compound represented by formula (III), and/or a polymer comprising at least one kind of polymerization unit of a fluoro-aliphatic group-containing monomer represented by formula (IV) or (V) and at least one kind of polymerization unit of an amide group-containing monomer represented by formula (VI):

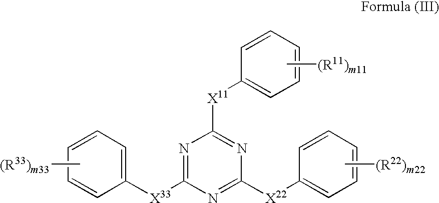

wherein R¹¹, R¹² and R¹³ each independently represent an alkoxy group having CF₃ group(s) or CF₂H group(s) at its terminal; X¹¹, X²² and X³³ each independently represent —NH—, —O— or —S—; and m¹¹, m²² and m³³ each independently represent an integer of 1 to 3;

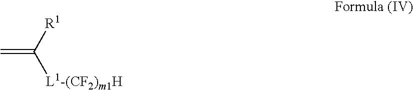

wherein R¹ represents a hydrogen atom, a halogen atom or a methyl group; L represents a divalent linking group; and m1 represents an integer of 1 to 18;

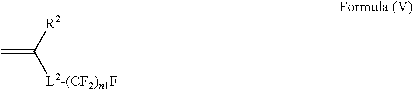

wherein R² represents a hydrogen atom, a halogen atom or a methyl group; L² represents a divalent linking group; and n1 represents an integer of 1 to 18; and

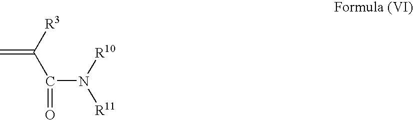

wherein R³ represents a hydrogen atom, a halogen atom or a methyl group; R¹⁰ and R¹¹ each independently represent a hydrogen atom, an alkyl group having 1 to 18 carbon atoms, an aromatic group having 6 to 20 carbon atoms, or a heterocyclic group having 1 to 20 carbon atoms; and R¹⁰ and R¹¹ may be bonded with each other to form a heterocyclic group.

<5> A light absorption anisotropic film formed by employing the dichroic dye composition described in any one of the above items <1> to <4>.
<6> The light absorption anisotropic film described in the above item <5>, wherein the dichroic dye in the light absorption anisotropic film is orientated with the tilt angle at the side of an alignment film of from 0° to 5°.
<7> A polarizing element comprising an alignment film and the light absorption anisotropic film described in the above item <5> or <6> on a support.
<8> The polarizing element described in the above item <7>, wherein the dichroic dye is orientated with the tilt angle at the side of the alignment film of from 0° to 5°.
<9> A liquid crystal display device comprising the light absorption anisotropic film described in the above item <5> or <6> or the polarizing element described in the above item <7> or <8>.
<10> A method of producing the polarizing element described in the above item <7> or <8>, comprising the steps of:

(1) rubbing a support or an alignment film formed on a support;

(2) applying the dichroic dye composition described in any one of the above items <1> to <4> dissolved in an organic solvent on the rubbing treated support or alignment film; and

(3) orientating the dichroic dye composition by causing the organic solvent to evaporate.

The present invention will be explained in detail below.

The dichroic dye composition of the present invention has a liquid crystallinity and is characterized in containing at least one kind of azo dye represented by formula (I). In the present invention, the term “dichroic dye” is defined as meaning a dye whose absorbing wavelength is different depending on the direction. Further, “dichroism” is calculated as a ratio of an absorbance of polarization in an absorption axis direction with respect to an absorbance of polarization in a polarization axis direction when the dichroic dye composition is used for the light absorption (optically) anisotropic film.

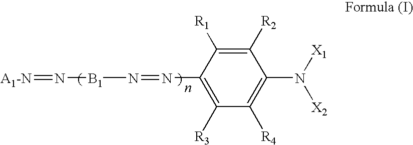

In formula (I), R₁, R₂, R₃, R₄, X₁ and X₂ each independently represent a hydrogen atom or a substituent; A₁ is a substituted or unsubstituted phenyl group, a substituted or unsubstituted naphthyl group, or a substituted or unsubstituted aromatic heterocyclic group; B₁ is a divalent substituted or unsubstituted aromatic hydrocarbon group or a divalent substituted or unsubstituted aromatic heterocyclic group; n represents an integer of 1 to 5; and at least one of B₁s represents a phenylene group having an alkyl group.

Hereinafter, the azo dye represented by formula (I) will be described in detail.

Examples of the substituents represented by R₁, R₂, R₃, R₄, X₁ and X₂ include an alkyl group (preferably an alkyl group having from 1 to 20, more preferably from 1 to 12, and particularly preferably from 1 to 8 carbon atoms, e.g., a methyl group, an ethyl group, an iso-propyl group, a tert-butyl group, an n-octyl group, an n-decyl group, an n-hexadecyl group, a cyclopropyl group, a cyclopentyl group, a cyclohexyl group), an alkenyl group (preferably an alkenyl group having from 2 to 20, more preferably from 2 to 12, and particularly preferably from 2 to 8 carbon atoms, e.g., a vinyl group, an allyl group, a 2-butenyl group, a 3-pentenyl group), an alkynyl group (preferably an alkynyl group having from 2 to 20, more preferably from 2 to 12, and particularly preferably from 2 to 8 carbon atoms, e.g., a propargyl group, a 3-pentynyl group), an aryl group (preferably an aryl group having from 6 to 30, more preferably from 6 to 20, and particularly preferably from 6 to 12 carbon atoms, e.g., a phenyl group, 2,6-diethylphenyl group, 3,5-di(trifluoromethyl)phenyl group, a naphthyl group, a biphenyl group), a substituted or unsubstituted amino group (preferably an amino group having from 0 to 20, more preferably from 0 to 10, and particularly preferably from 0 to 6 carbon atoms, e.g., an unsubstituted amino, a methylamino group, a dimethylamino group, a diethylamino group, an anilino group), an alkoxy group (preferably an alkoxy group having from 1 to 20, more preferably from 1 to 10, and particularly preferably from 1 to 6 carbon atoms, e.g., a methoxy group, an ethoxy group, a butoxy group), an alkoxycarbonyl group (preferably an alkoxycarbonyl group having from 2 to 20, more preferably from 2 to 10, and particularly preferably from 2 to 6 carbon atoms, e.g., a methoxycarbonyl group, an ethoxycarbonyl group), an acyloxy group (preferably an acyloxy group having from 2 to 20, more preferably from 2 to 10, and particularly preferably from 2 to 6 carbon atoms, e.g., an acetoxy group, a benzoyloxy group), an acylamino group (preferably an acylamino group having from 2 to 20, more preferably from 2 to 10, and particularly preferably from 2 to 6 carbon atoms, e.g., an acetylamino group, a benzoylamino group), an alkoxycarbonylamino group (preferably an alkoxycarbonylamino group having from 2 to 20, more preferably from 2 to 10, and particularly preferably from 2 to 6 carbon atoms, e.g., a methoxycarbonylamino group), an aryloxycarbonylamino group (preferably an aryloxycarbonylamino group having from 7 to 20, more preferably from 7 to 16, and particularly preferably from 7 to 12 carbon atoms, e.g., a phenyloxycarbonylamino group), a sulfonylamino group (preferably a sulfonylamino group having from 1 to 20, more preferably from 1 to 10, and particularly preferably from 1 to 6 carbon atoms, e.g., a methanesulfonylamino group, a benzenesulfonylamino group), a sulfamoyl group (preferably a sulfamoyl group having from 0 to 20, more preferably from 0 to 10, and particularly preferably from 0 to 6 carbon atoms, e.g., a sulfamoyl group, a methylsulfamoyl group, a dimethylsulfamoyl group, a phenylsulfamoyl group), a carbamoyl group (preferably a carbamoyl group having from 1 to 20, more preferably from 1 to 10, and particularly preferably from 1 to 6 carbon atoms, e.g., an unsubstituted carbamoyl group, a methylcarbamoyl group, a diethylcarbamoyl group, a phenylcarbamoyl group), an alkylthio group (preferably an alkylthio group having from 1 to 20, more preferably from 1 to 10, and particularly preferably from 1 to 6 carbon atoms, e.g., a methylthio group, an ethylthio group), an arylthio group (preferably an arylthio group having from 6 to 20, more preferably from 6 to 16, and particularly preferably from 6 to 12 carbon atoms, e.g., a phenylthio group), a sulfonyl group (preferably a sulfonyl group having from 1 to 20, more preferably from 1 to 10, and particularly preferably from 1 to 6 carbon atoms, e.g., a mesyl group, a tosyl group), a sulfinyl group (preferably a sulfinyl group having from 1 to 20, more preferably from 1 to 10, and particularly preferably from 1 to 6 carbon atoms, e.g., a methanesulfinyl group, a benzenesulfinyl group), a ureido group (preferably a ureido group having from 1 to 20, more preferably from 1 to 10, and particularly preferably from 1 to 6 carbon atoms, e.g., an unsubstituted ureido group, a methylureido group, a phenylureido group), a phosphoric acid amido group (preferably a phosphoric acid amido group having from 1 to 20, more preferably from 1 to 10, and particularly preferably from 1 to 6 carbon atoms, e.g., a diethylphosphoric acid amido group, a phenylphosphoric acid amido group), a hydroxy group, a mercapto group, a halogen atom (e.g., a fluorine atom, a chlorine atom, a bromine atom, or an iodine atom), a cyano group, a nitro group, a hydroxamic acid group, a sulfino group, a hydrazino group, an imino group, a heterocyclic group (preferably a heterocyclic group having from 1 to 30, and more preferably from 1 to 12 carbon atoms; containing, as a hetero atom(s), for example, a nitrogen atom, an oxygen atom, or a sulfur atom, and specifically, e.g., an imidazolyl group, a pyridyl group, a quinolyl group, a furyl group, a piperidyl group, a morpholino group, a benzoxazolyl group, a benzimidazolyl group, a benzthiazolyl group can be exemplified), and a silyl group (preferably a silyl group having 3 to 40, more preferably 3 to 30, and particularly preferably 3 to 24 carbon atoms, e.g. a trimethylsilyl group, a triphenylsilyl group).

These substituents may further be substituted. When two or more substituents are present, the substituents may be the same as or different from each other. Alternatively, they may bind to each other, forming a ring, if possible.

R₁ to R₄ each are preferably a hydrogen atom, an alkyl group, an alkoxy group or a halogen atom; more preferably a hydrogen atom, an alkyl group or an alkoxy group; and most preferably a hydrogen atom. X₁ and X₂ each are preferably a hydrogen atom or an alkyl group; and most preferably an alkyl group.

A₁ is a substituted or unsubstituted phenyl group, a substituted or unsubstituted naphthyl group, or a substituted or unsubstituted aromatic heterocyclic group.

With regard to the substituent which may be possessed by the phenyl group or the naphthyl group, a group which is introduced in order to raise a solubility of the azo compound, a group having an electron donative property or an electron withdrawing property which is introduced in order to adjust color tone as a dye, or a group having the polymerizable group which is introduced in order to fixate an orientation is preferable. Specific examples include a substituent represented by R₁, R₂, R₃, R₄, X₁ and X₂. Preferred examples of the substituent include a substituted or unsubstituted alkyl group, a substituted or unsubstituted alkenyl group, a substituted or unsubstituted alkynyl group, a substituted or unsubstituted aryl group, a substituted or unsubstituted alkoxy group, a substituted or unsubstituted alkoxycarbonyl group, a substituted or unsubstituted acyloxy group, a substituted or unsubstituted acylamino group, a substituted or unsubstituted amino group, a substituted or unsubstituted alkoxycarbonylamino group, a substituted or unsubstituted sulfonylamino group, a substituted or unsubstituted sulfamoyl group, a substituted or unsubstituted carbamoyl group, a substituted or unsubstituted alkylthio group, a substituted or unsubstituted sulfonyl group, a substituted or unsubstituted ureido group, a nitro group, a hydroxy group, a cyano group and a halogen atom.

The alkyl group is an alkyl group having preferably from 1 to 20 carbon atoms, more preferably from 1 to 12 carbon atoms. Examples of the group which may be substituted on the alkyl group include an alkoxy group, an acyloxy group, a hydroxy group and a halogen atom.

The group which may be substituted on the alkyl group is preferably a polymerizable group. The polymerization reaction of the polymerizable group, but not to be limited to, is preferably addition polymerization (including ring opening polymerization) or condensation polymerization. In other words, the polymerizable group is preferably a polymerizable group capable of addition polymerization reaction or condensation polymerization reaction.

Specific examples of the polymerizable group are shown below, but the invention is not meant to be limited to these.

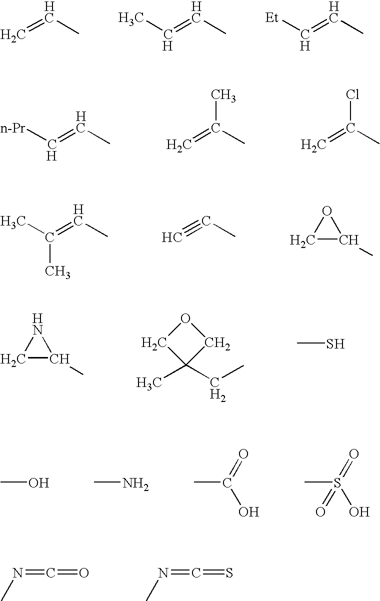

The polymerizable group is preferably a polymerizable group capable of a radical polymerization or a cationic polymerization. General radically polymerizable group can be used as the radically polymerizable group and a (meta)acrylate group is preferable. General cationically polymerizable group can be used as the cationically polymerizable group. Specific examples include an alicyclic ether group, a cyclic acetal group, a cyclic lactone group, a cyclic thioether group, a spiro orthoester group, and a vinyloxy group. Among those, the alicyclic ether group and the vinyloxy group are preferred; and an epoxy group, an oxetanyl group, and a vinyloxy group are particularly preferable.

The alkenyl group is an alkenyl group having preferably from 2 to 20 carbon atoms, more preferably from 2 to 12 carbon atoms. The group which may be substituted on the alkenyl group is synonymous with the group which may be substituted on the alkyl group and the preferable examples are also the same.

The alkynyl group is an alkynyl group having preferably from 2 to 20 carbon atoms, more preferably from 2 to 12 carbon atoms. The group which may be substituted on the alkynyl group is synonymous with the group which may be substituted on the alkyl group and the preferable examples are also the same.

The aryl group is an aryl group having preferably from 6 to 20 carbon atoms, more preferably from 6 to 12 carbon atoms. The group which may be substituted on the aryl group is synonymous with the group which may be substituted on the alkyl group and the preferable examples are also the same.

The alkoxy group is an alkoxy group having preferably from 1 to 20 carbon atoms, more preferably from 1 to 12 carbon atoms. The group which may be substituted on the alkoxy group is synonymous with the group which may be substituted on the alkyl group and the preferable examples are also the same.

The alkoxycarbonyl group is an alkoxycarbonyl group having preferably from 2 to 20 carbon atoms, more preferably from 2 to 12 carbon atoms. The group which may be substituted on the alkoxycarbonyl group is synonymous with the group which may be substituted on the alkyl group and the preferable examples are also the same.

The acyloxy group is an acyloxy group having preferably from 2 to 20 carbon atoms, more preferably from 2 to 12 carbon atoms. The group which may be substituted on the acyloxy group is synonymous with the group which may be substituted on the alkyl group and the preferable examples are also the same.

The amino group is an amino group having preferably from 1 to 20 carbon atoms, more preferably from 1 to 12 carbon atoms. The group which may be substituted on the amino group is synonymous with the group which may be substituted on the alkyl group and the preferable examples are also the same.

The acylamino group is an acylamino group having preferably from 1 to 20 carbon atoms, more preferably from 1 to 12 carbon atoms. The group which may be substituted on the acylamino group is synonymous with the group which may be substituted on the alkyl group and the preferable examples are also the same.

The alkoxycarbonylamino group is an acylamino group having preferably from 2 to 20 carbon atoms, more preferably from 2 to 12 carbon atoms. The group which may be substituted on the alkoxycarbonylamino group is synonymous with the group which may be substituted on the alkyl group and the preferable examples are also the same.

The sulfonylamino group is a sulfonylamino group having preferably from 1 to 20 carbon atoms, more preferably from 1 to 12 carbon atoms. The group which may be substituted on the sulfonylamino group is synonymous with the group which may be substituted on the alkyl group and the preferable examples are also the same.

The sulfamoyl group is a sulfonylamino group having preferably from 1 to 20 carbon atoms, more preferably from 1 to 12 carbon atoms. The group which may be substituted on the sulfamoyl group is synonymous with the group which may be substituted on the alkyl group and the preferable examples are also the same.

The group which may be substituted on the carbamoyl group is synonymous with the group which may be substituted on the alkyl group and the preferable examples are also the same.

The alkylthio group is an alkylthio group having preferably from 1 to 20 carbon atoms, more preferably from 1 to 12 carbon atoms. The group which may be substituted on the alkylthio group is synonymous with the group which may be substituted on the alkyl group and the preferable examples are also the same.

The sulfonyl group is a sulfonyl group having preferably from 1 to 20 carbon atoms, more preferably from 1 to 12 carbon atoms. The group which may be substituted on the sulfonyl group is synonymous with the group which may be substituted on the alkyl group and the preferable examples are also the same.

The ureido group is an ureido group having preferably from 2 to 20 carbon atoms, more preferably from 2 to 12 carbon atoms. The group which may be substituted on the ureido group is synonymous with the group which may be substituted on the alkyl group and the preferable examples are also the same.

The phenyl group or the naphthyl group may have these substituents in numbers of 1 to 5, preferably 1 or 2.

The aromatic heterocyclic group is preferably a group having a heterocyclic origin of single ring or double rings. Examples of the atom excluding a carbon atom which composes the aromatic heterocyclic group include a nitrogen atom, a sulfur atom and an oxygen atom. When the aromatic heterocyclic group has a plurality of atom excluding the carbon atom, those atoms may be the same with, or different from each other. Specific examples of the aromatic heterocyclic group include a pyridyl group, a quinolyl group, a thiazolyl group, a benzothiazolyl group, a quinolonyl group, a naphthalimidoyl group, and a group having a heterocyclic origin as shown below.

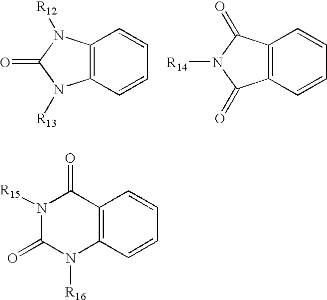

R₁₂, R₁₃, R₁₄, R₁₅ and R₁₆ each independently represent a hydrogen atom, a substituted or unsubstituted alkyl group, or a substituted or unsubstituted phenyl group. The substituent on the alkyl group and the phenyl group is synonymous with the substituent which may be substituted on the alkyl group and the preferable examples are also the same.

A pyridyl group, a quinolyl group or a phthalimide-yl group is preferable as the aromatic heterocyclic group.

A₁ is particularly preferably a substituted or unsubstituted phenyl group.

B₁ represents a divalent substituted or unsubstituted aromatic hydrocarbon group or a divalent substituted or unsubstituted aromatic heterocyclic group, with the proviso that at least one B₁ among nB₁'s represents a phenylene group having an alkyl group. The alkyl group in this occasion is an alkyl group preferably having 1 to 20 carbon atoms, more preferably having 1 to 12 carbon atoms and particularly preferably having 1 to 8 carbon atoms; examples include a methyl group, an ethyl group, an isopropyl group, a tert-butyl group, an n-octyl group, an n-decyl group, an n-hexadecyl group, a cyclopropyl group, a cyclopentyl group, and a cyclohexyl group. Particularly preferable examples are the methyl group and the ethyl group, and the most preferable example is the methyl group.

The aromatic hydrocarbon represented by B₁ is preferably a phenylene group or a naphthylene group. Examples of the substituent which the aromatic hydrocarbon may have include a substituted or unsubstituted alkyl group, a substituted or unsubstituted alkoxy group, a hydroxy group, a nitro group, a halogen atom, a substituted or unsubstituted amino group, a substituted or unsubstituted acylamino group, and a cyano group. Additionally, a preferable number of carbon atoms and the substituent which may be possessed in the substituted alkyl group, the substituted alkoxy group, the substituted amino group and the substituted acylamino group are synonymous with that described in the case of the above-mentioned A₁ being the phenyl group or the naphthyl group, and the preferable examples are also the same.

The aromatic heterocyclic group represented by B₁ is preferably a group having a heterocyclic origin of single ring or double rings. Examples of the atom composing the aromatic heterocyclic group excluding the carbon atom include a nitrogen atom, a sulfur atom and an oxygen atom, and the nitrogen atom is particularly preferable. When the aromatic heterocyclic group has a plurality of atom excluding the carbon atom, those atoms may be the same with, or different from each other. Specific examples of the aromatic heterocyclic group include a pyridinediyl group, a quinolinediyl group, an isoquinolinediyl group, a benzothiadiazolediyl group, and a phthalimidediyl group. Among those, the quinolinediyl group and the isoquinolinediyl group are preferable.

Examples of the substituent which may be possessed by the aromatic heterocyclic group include an alkyl group such as a methyl group and an ethyl group, an alkoxy group such as a methoxy group and an ethoxy group, an amino group such as an unsubstituted amino group and a methylamino group, an acetylamino group, an acylamino group, a nitro group, a hydroxy group, a cyano group and a halogen atom.

B₁ is particularly preferably a divalent substituted or unsubstituted phenylene group.

n represents an integer of 1 to 5, and preferably in integer of 2 to 4.

The azo dye represented by formula (I) is particularly preferably the azo dye represented by formula (II).

The azo dye represented by formula (II) is described below.

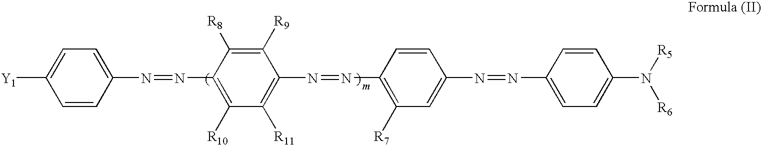

In formula (II), R₅, R₆ and R₇ each independently represent an alkyl group; R₈, R₉, R₁₀ and R₁₁ each independently represent a hydrogen atom or a substituent; Y₁ represents a substituted or unsubstituted, alkyl group, alkenyl group, alkynyl group, aryl group, alkoxy group, alkoxycarbonyl group, acyloxy group, acylamino group, alkoxycarbonylamino group, sulfonylamino group, sulfamoyl group, carbamoyl group, alkylthio group, sulfonyl group or ureido group; and m represents an integer of 1 to 3.

The alkyl group represented by R₅, R₆ and R₇ is synonymous with the alkyl group described as X₁ and X₂ in formula (I), and the preferred examples are also the same. The alkyl group represented by R₅, R₆ and R₇ is preferably a methyl group or an ethyl group. The alkyl group represented by R₇ is most preferably a methyl group.

The substituent represented by R₈, R₉, R₁₀ and R₁₁ is synonymous with the substituent of B₁ in formula (I), and the preferred examples are also the same.

In formula (II), it is most preferable that R₅ and R₆ each are a methyl group or an ethyl group, R₇ is a methyl group, R₈, R₉, R₁₀ and R₁₁ each are a hydrogen atom, Y₁ is an alkyl group having 1 to 8 carbon atoms, and m is 1.

The substituted or unsubstituted alkyl group, alkenyl group, alkynyl group, aryl group, alkoxy group, alkoxycarbonyl group, acyloxy group, acylamino group, alkoxycarbonylamino group, sulfonylamino group, sulfamoyl group, carbamoyl group, alkylthio group, sulfonyl group or ureide group represented by Y₁ is synonymous with the alkyl group, the alkenyl group, the alkynyl group, the aryl group, the alkoxy group, the alkoxycarbonyl group, the acyloxy group, the acylamino group, the alkoxycarbonylamino group, the sulfonylamino group, the sulfamoyl group, the carbamoyl group, the alkylthio group, the sulfonyl group or the ureide group which is a substituent of the group represented by A₁ in formula (I), and the preferred examples are also the same. Y₁ is preferably an alkyl group, an aryl group, an alkoxy group, an alkoxycarbonyl group, an acyloxy group, an acylamino group, a sulfonylamino group, a sulfamoyl group, a carbamoyl group, an alkylthio group or a sulfonyl group; more preferably an alkyl group, an aryl group, an alkoxy group, an alkoxycarbonyl group, an acyloxy group or an alkylthio group; and most preferably an alkyl group, an aryl group or an alkoxy group.

The specific examples of the azo dye represented by formula (I) are shown below. However, the present invention is not limited to these specific examples.

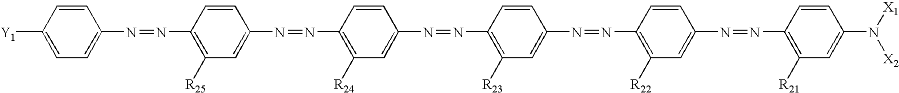

| No. | X₁ | X₂ | R₂₁ | R₂₂ | R₂₃ | R₂₄ | R₂₅ | Y₁ |
|---|---|---|---|---|---|---|---|---|
| A-1 | —C₂H₅ | —C₂H₅ | —H | —CH₃ | —H | —H | —H | —C₄H₉ |
| A-2 | —C₂H₅ | —C₂H₅ | —H | —CH₃ | —CH₃ | —CH₃ | —H | —C₄H₉ |
| A-3 | —CH₃ | —CH₃ | —H | —CH₃ | —H | —H | —H | —C₄H₉ |

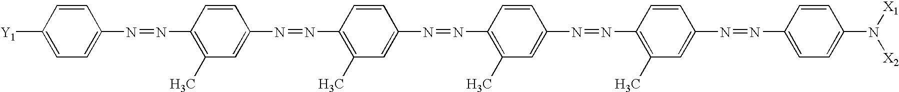

| No. | X₁ | X₂ | Y₁ |
|---|---|---|---|
| A-4 | —C₂H₅ | —C₂H₅ | 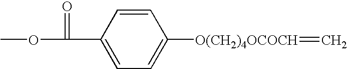 |
| A-5 | —C₂H₅ | —C₂H₅ | 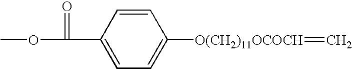 |
| A-6 | —CONH(CH₂)₂OCOC(CH₃)═CH₂ | —H | —C₄H₉ |
| A-7 | —CONH(CH₂)₂OCOC(CH₃)═CH₂ | —C₂H₅ | —C₄H₉ |
| A-8 | —CONH(CH₂)₂OCOC(CH₃)═CH₂ | —C₂H₅ | 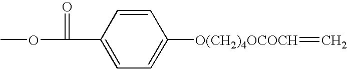 |

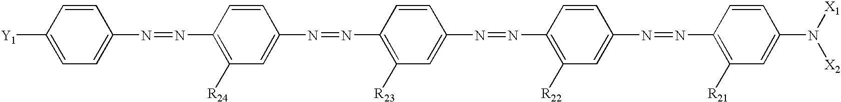

| No. | X₁ | X₂ | R₂₁ | R₂₂ | R₂₃ | R₂₄ | Y₁ |
|---|---|---|---|---|---|---|---|
| A-9 | —C₂H₅ | —C₂H₅ | —H | —CH₃ | —H | —H | —C₄H₉ |
| A-10 | —C₂H₅ | —C₂H₅ | —CH₃ | —CH₃ | —H | —H | —C₄H₉ |
| A-11 | —C₂H₅ | —C₂H₅ | —H | —CH₃ | —CH₃ | —CH₃ | —C₄H₉ |
| A-12 | —CONH(CH₂)₂OCOC(CH₃)═CH₂ | —H | —H | —CH₃ | —H | —H | —C₄H₉ |
| A-13 | —CONH(CH₂)₂OCOC(CH₃)═CH₂ | —C₂H₅ | —H | —CH₃ | —H | —H | —C₄H₉ |
| A-14 | —CONH(CH₂)₂OCOC(CH₃)═CH₂ | —C₂H₅ | —H | —CH₃ | —CH₃ | —CH₃ | 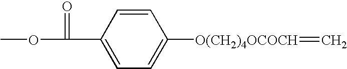 |
| A-15 | —C₂H₅ | —C₂H₅ | —H | —CH₃ | —CH₃ | —CH₃ | 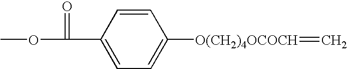 |

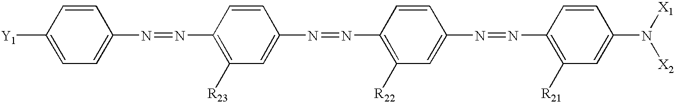

| No. | X₁ | X₂ | R₂₁ | R₂₂ | R₂₃ | Y₁ |
|---|---|---|---|---|---|---|
| A-16 | —C₂H₅ | —C₂H₅ | —H | —CH₃ | —H | —C₄H₉ |
| A-17 | —C₂H₅ | —C₂H₅ | —H | —CH₃ | —CH₃ | —C₄H₉ |
| A-18 | —C₂H₅ | —C₂H₅ | —H | —CH₃ | —H | 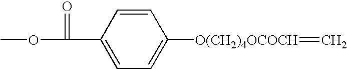 |
| A-19 | —C₂H₅ | —C₂H₅ | —H | —CH₃ | —H | 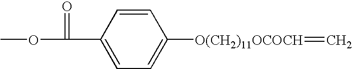 |
| A-20 | —CONH(CH₂)₂OCOC(CH₃)═CH₂ | —H | —H | —CH₃ | —H | —C₄H₉ |
| A-21 | —CONH(CH₂)₂OCOC(CH₃)═CH₂ | —C₂H₅ | —H | —CH₃ | —H | —C₄H₉ |
| A-22 | —CONH(CH₂)₂OCOC(CH₃)═CH₂ | —C₂H₅ | —H | —CH₃ | —H | 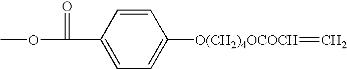 |
| A-23 | —CONH(CH₂)₂OCOC(CH₃)═CH₂ | —C₂H₅ | —H | —CH₃ | —H | 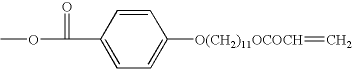 |
| A-24 | —C₂H₅ | —C₂H₅ | —OCH₃ | —CH₃ | —H | —C₄H₉ |
| A-25 | —C₂H₅ | —C₂H₅ | —H | —CH₃ | —CH₃ | 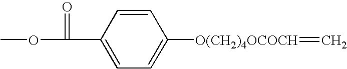 |
| A-26 | —CONH(CH₂)₂OCOC(CH₃)═CH₂ | —C₂H₅ | —H | —CH₃ | —CH₃ | 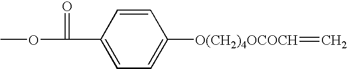 |

| | |
|---|---|
| | A-27 |
| 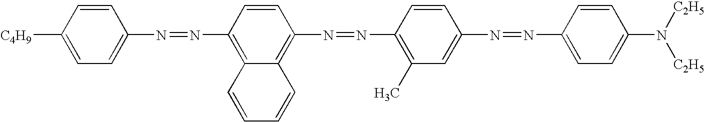 | |
| | A-28 |
| 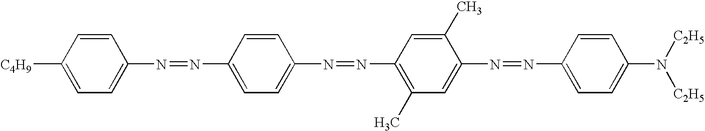 | |
| | A-29 |
| 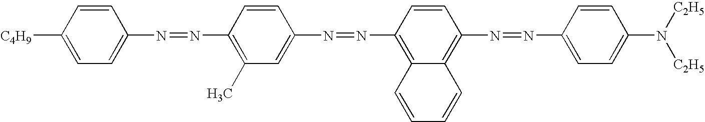 | |
| | A-30 |
| 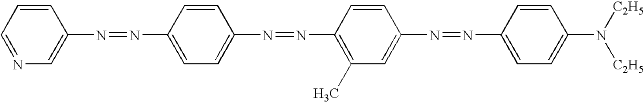 | |
| | A-31 |
| 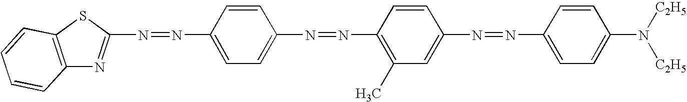 | |
| | A-32 |
| 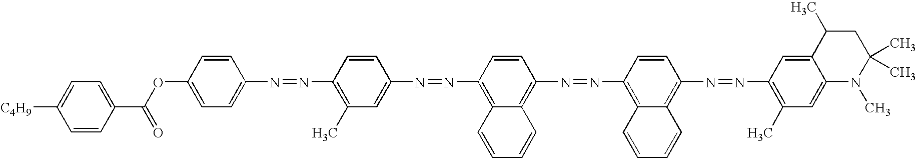 | |
| | A-33 |
| 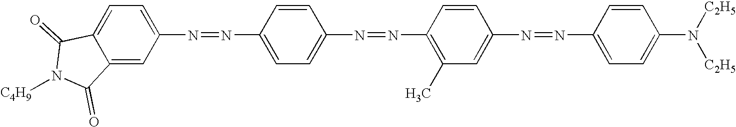 | |
| | A-34 |
| 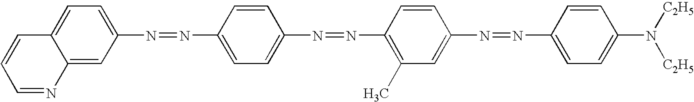 | |
| | A-35 |
| 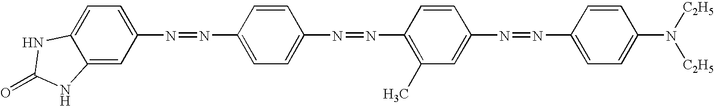 | |
| | A-36 |
| 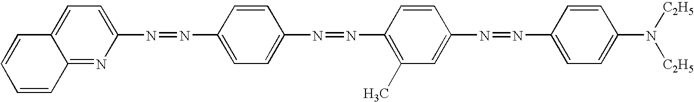 | |
| | A-37 |
| 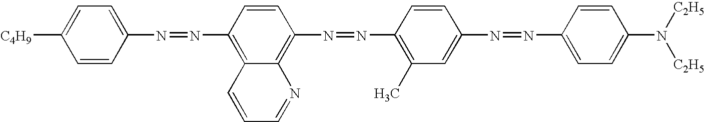 | |

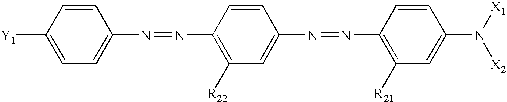

| No. | X₁ | X₂ | R₂₁ | R₂₂ | Y₁ |
|---|---|---|---|---|---|
| A-38 | —C₂H₅ | —C₂H₅ | —H | —CH₃ | 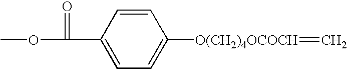 |
| A-39 | —CONH(CH₂)₂OCOC(CH₃)═CH₂ | —C₂H₅ | —H | —CH₃ | 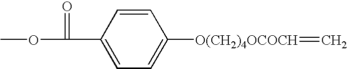 |
| A-40 | —C₂H₅ | —C₂H₅ | —H | —CH₃ | —C₄H₉ |

| | |
|---|---|
| | A-41 |
| 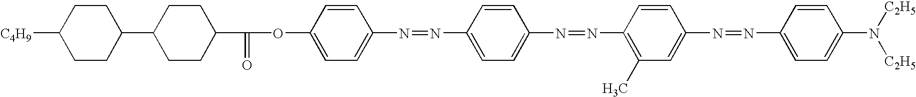 | |
| | A-42 |
| 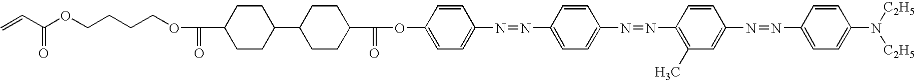 | |
| | A-43 |
| 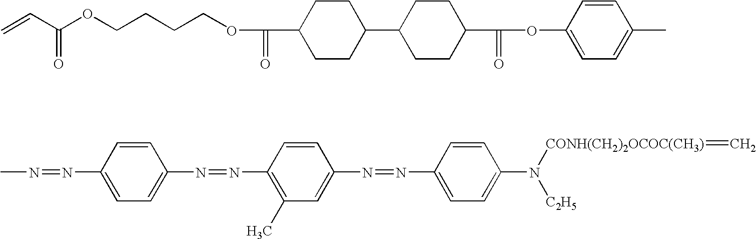 | |
| | A-44 |
| 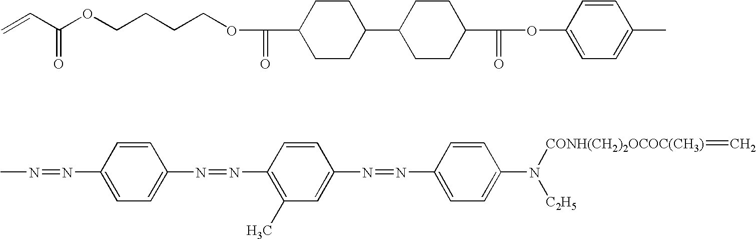 | |
| | A-45 |
| 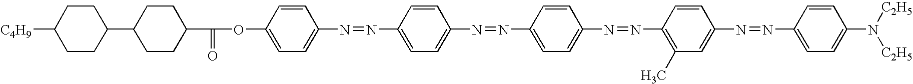 | |
| 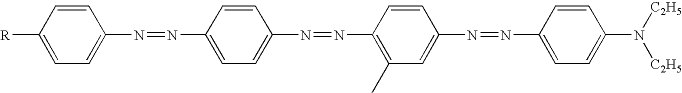 | |
| | A-46 |
| R = —CF₃ | |
| | A-47 |
| R = —C₈H₁₇ | |
| | A-48 |
| R = —COOH | |
| | A-49 |
| R = —COOCH₃ | |
| | A-50 |
| R = —COOC₂H₅ | |
| | A-51 |
| R = COOC₆H₁₃ | |
| | A-52 |
| 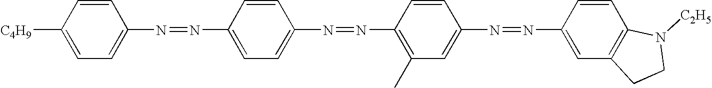 | |
| | A-53 |
| 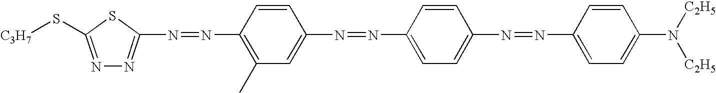 | |
| | A-54 |
| 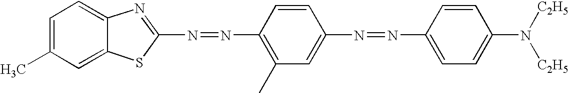 | |
| | A-55 |
| 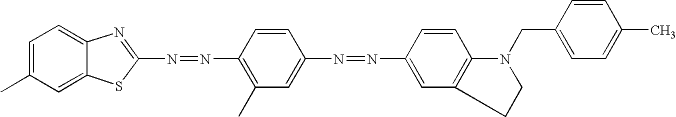 | |
| | A-56 |
| 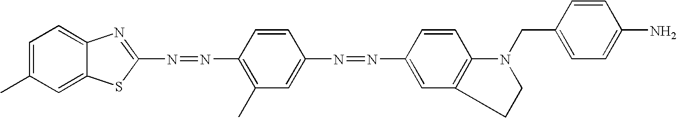 | |
| | A-57 |
| 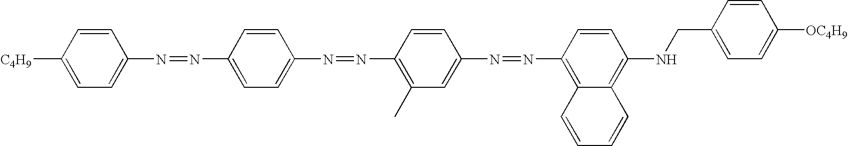 | |

The azo dye represented by formula (I) or (II) can be prepared according to the method described in, for example, Journal of Materials Chemistry (1999), 9(11), pp. 2755-2763.

The azo dye represented by the formula (I) or (II) has, as apparent from its molecular structure, a planar molecular shape and a favorable linearity, further having a rigid and solid core part and a flexible side-chain part, and also has a polar amino group at its terminal of molecular long axis of the azo dye. Accordingly, it has a property easily revealing a liquid crystallinity, especially a nematic liquid crystallinity. Furthermore, a strong intermolecular interaction acts due to the high flatness of the molecule, and it also has a property of easily forming an association state of the molecules each other.

The dichroic dye composition containing the azo dye represented by formula (I) or (II) of the present invention not only reveals the high absorbance in a wide visible wavelength region caused by association formation, but also has a nematic liquid crystallinity. Accordingly, for example, due to passing through a lamination process such as coating over the surface of an alignment film (orientating film) after rubbing, a high level of molecular orientation state is realizable. Therefore, an employment of the dichroic dye composition containing the azo dye represented by formula (I) or (II) of the present invention as a light absorption anisotropic film enables to produce a polarizing element with high polarizing property.

In the present invention, only a single azo dye represented by formula (I) or (II) may be used, or two or more azo dyes may be used in combination. Or a combination of the azo dye in the present invention and another dye compound may be also adequate. Examples of the another dye compound include an azo dye employed except in the present invention, a cyanine series dye, an azo metal complex, a phthalocyanine series dye, a pyrylium series dye, a thiopyrylium series dye, an azulenium series dye, a squarylium series dye, a naphthoquinone series dye, a triphenylmethane series dye and a triallyl methane series dye.

The light absorption anisotropic film of the present invention is formed by employing, as a main component, the dichroic dye, and the content of the dichroic dye contained in the dichroic dye composition of the present invention is 70 mass % or more, particularly preferably 80 mass % or more, and the most preferably 90 mass % or more.

Further, the content of the azo dye represented by formula (I) or (II) is preferably 50 mass % or more with respect to the dichroic dye, and particularly preferably 70 mass % or more.

Furthermore, although the content of the azo dye represented by formula (I) or (II) contained in the dichroic dye composition of the present invention is not limited in particular, it is preferably 70 mass % or more, particularly preferably 80 mass % or more.

### [Additives for Light Absorption Anisotropic Film]

Any additive may be used in the dichroic dye composition of the present invention in combination with the above dichroic dye. Examples of the additive include an anti-unevenness-by-wind agent, an anti-cissing agent, an additive to control the tilt angle of an alignment film (tilt angle of the dichroic dye at the interface of the light absorption anisotropic film/the alignment film), an additive to control the tilt angle of air interface (tilt angle of the dichroic dye at the interface of the light absorption anisotropic film/air), a polymerization initiator, an additive (plasticizers) for decreasing an orientation temperature, polymerizable monomer, saccharides, and a chemical agent or so having at least any function of an antifungal activity, an antibacterial activity and a sterilization activity. In the following, a description will be made about each additive.

### [Anti-Unevenness-by-Wind Agent]

Fluorine based polymers are suitably employable in general as a material for preventing unevenness by wind in a coating process used together with the dichroic dye. The fluorine based polymers to be used are riot particularly limited so long as not furiously obstruct a tilt angle change or orientation of the dichroic dye. JP-A-2004-198511, JP-A-2004-333852, JP-A-2005-179636 and JP-A-2005-206638 disclose about examples of the fluorine based polymer usable as the anti-unevenness-by-wind agent. Using fluorine based polymer together with the dichroic dye enables to display images of high display quality without generating the unevenness. Further, coating properties such as a cissing or so can be also improved. The addition amount of the fluorine based polymer used for the purpose of preventing the unevenness by wind without disturbing the orientation of the dichroic dye is, in general, preferably within the range of 0.1 to 2 mass % with respect to the dichroic dye; more preferably within the range of 0.1 to 1 mass %, and furthermore preferably within the range of 0.4 to 1 mass %.

### [Anti-Cissing Agent]

Polymers are usually used as a material for preventing cissing while coating. Any polymers, which can be mixed with the dichroic dye compatibly, can be used unless they change the tilt angle of the dichroic dye or inhibit alignment of the dichroic dye substantially. Examples of the polymer, which can be used as an anti-cissing agent, include the polymers disclosed in JP-A-8-95030, and especially preferred examples of the polymer include cellulose esters. Examples of the cellulose ester include cellulose acetate, cellulose acetate propionate, hydroxypropyl cellulose and cellulose acetate butyrate. Preventing the anti-cissing agent from inhibiting alignment of the dichroic dye, in usual, the amount of the polymer as the anti-cissing agent is preferably from 0.1 to 10 mass %, more preferably from 0.1 to 8 mass % and much more preferably from 0.1 to 5 mass % with respect to the total weight of the dichroic dye composition.

### [Agent for Controlling Tilt Angle of Alignment Film]

Any compound having both of a polar group and a non-polar group may be added for controlling a tilt angle of an alignment film. Examples of the compound having both of a polar group and a non-polar group include P^{O}—OH, P^{O}—COOH, P^{O}—O—P^{O}, P^{O}—NH₂, P^{O}—NH—P^{O}, P^{O}—SH, P^{O}—S—P^{O}, P^{O}—CO—P^{O}, P^{O}—COO—P^{O}, P^{O}—CONH—P^{O}, P^{O}—CONHCO—P^{O}, P₁—SO₃H, P^{O}—SO₃—P^{O}, P^{O}—SO₂NH—P^{O}, P^{O}—SO₂NHSO₂—P^{O}, P^{O}—C═N—P^{O}, HO—P(—OP^{O})₂, (HO—)₂P^{O}—OP^{O}, P(—OR)₃, HO—P^{O}(—OP^{O})₂, (HO—)₂P^{O}—OP^{O}, P^{O}(—OP^{O})₃, P^{O}—NO₂ and P^{O}—CN; and organic salts thereof. Examples of the organic salts include organic salts of the above-described compound such as ammoniums, carboxylates, sulfonates; and pyridinium salts. Among these, P^{O}—OH, P^{O}—COOH, P^{O}—O—P^{O}, P^{O}—NH₂, P₁—SO₃H, HO—P^{O}(—OP^{O})₂, (HO—)₂P^{O}—OP^{O}, P^{O}(—OP^{O})₃ and organic salts thereof are preferred. Herein, P^{O} represents a non-polar group. When there are plurality of P⁰, each P⁰ may be the same with, or different from each other.

Examples of P^{O} include an alkyl group (preferably a linear, branched or cyclic, substituted or unsubstituted alkyl group having 1 to 30 carbon atoms), an alkenyl group (preferably a linear, branched or cyclic, substituted or unsubstituted alkenyl group having 1 to 30 carbon atoms), an alkynyl group (preferably a linear, branched or cyclic, substituted or unsubstitlted alkynyl group having 1 to 30 carbon atoms), an aryl group (preferably a substituted or unsubstituted aryl group having 6 to 30 carbon atoms) and a silyl group (preferably a substituted or unsubstituted silyl group having 3 to 30 carbon atoms). The non-polar group may have a substituent such as a halogen atom, an alkyl group (whose meaning includes a cycloalkyl group such as a monocyclo or bicyclo alkyl group), an alkenyl group (whose meaning include a cycloalkenyl group such as monocyclo or bicyclo alkenyl group), an alkynyl group, an aryl group, a heterocyclic group, a cyano group, a hydroxyl group, a nitro group, a carboxyl group, an alkoxy group, an aryloxy group, a silyloxy group, a heterocyclic oxy group, an acyloxy group, a carbamoyloxy group, an alkoxycarbonyloxy group, aryloxycarbonyloxy group, an amino group (whose meaning includes an anilino group), an acylamino group, an aminocarbonylamino group, an alkoxycarbonylamino group, an aryloxycarbonylamino group, a sulfamoylamino group, an alkylsulfonylamino group, an arylsulfonylamino group, a mercapto group, an alkylthio group, an arylthio group, a heterocyclic thio group, a sulfamoyl group, a sulfo group, an alkylsulfinyl group, an arylsulfinyl group, an alkylsulfonyl group, an arylsulfonyl group, an acyl group, an aryloxycarbonyl group, an alkoxycarbonyl group, a carbamoyl group, an arylazo group, a heterocyclic azo group, an imido group, a phosphino group, a phosphinyl group, a phosphinyloxy group, a phosphinylamino group and a silyl group.

In the present invention, adding an agent for controlling a tilt angle of an alignment film into the dichroic dye composition coating solution and orientating the dichroic dye in the presence of the agent for controlling a tilt angle of an alignment film enable to adjust the tilt angle of the dichroic dye at an alignment film interface. The addition amount of the agent for controlling a tilt angle of an alignment film is, in general, preferably from 0.0001 mass % to 30 mass % with respect to the mass of the dichroic dye, more preferably from 0.001 mass % to 20 mass %, and further more preferably from 0.005 mass % to 10 mass %. In the present invention, an agent for controlling a tilt angle of an alignment film disclosed in JP-A-2006-58801 are usable.

### [Polymerization Initiator]

It is preferable to form the light absorption anisotropic film by fixing oriented state of the dichroic dye, and it is also preferable to fix the dichroic dye by utilizing the polymerization reaction. Examples of polymerization reactions which can be used in the present invention include thermal polymerization reactions employing thermal polymerization initiators and photo-polymerization reactions employing photo-polymerization initiators. Photo-polymerization reactions are preferred to avoid a deformation or a degradation of a support of the anisotropic layer. It is possible to refer to descriptions from paragraph Nos. [0050] to [0051] in JP-A-2001-91741 with respect to various matters of the polymerization initiator such as examples of the polymerization initiator, a proper amount of the polymerization initiator to be used or proper photo-irradiation energy for polymerization.

### [Polymerizable Monomer]

Any polymerizable monomer may be used with the dichroic dye. Any polymerizable monomers, which can be mixed with the dichroic dye compatibly, can be used unless they contribute to varying a tile angle of the dichroic dye or inhibiting an alignment of the dichroic dye substantially. Among them, a compound having an ethylenically unsaturated group such as a vinyl group, a vinyloxy group, an acryloyl group or a methacryloyl group, may be preferably used. In usual, the amount of the polymerizable monomer is preferably from 1 to 50 mass %, and more preferably from 5 to 30 mass %, with respect to the total weight of the dichroic dye. When a polymerizable monomer having two or more reactive functional groups is used with the dichroic dye, the adhesion property between the light absorption anisotropic layer and the alignment film is improved.

### [Saccharides]

Saccharides may be added into the composition of the present invention. The addition of the saccharides will enhance the association degree of a dye association, and will be able to elevate a molecular orientation of the dye as a result.

Examples of the saccharides include monosaccharides, disaccharides, polysaccharides and derivatives of them such as sugar alcohol. For the purpose of revealing the effect of the present invention, it is preferable that the number of hydroxy groups in the saccharides is usually 2 or more, preferably 3 or more and 18 or less, further preferably 12 or less in the viewpoint of the molecular association property. When the hydroxy group is too many, it is not preferable because mutual action with the dye becomes so strong that the hydroxy group precipitates and deteriorates the orientation of the dye film. When the hydroxy group is too few, it is also not preferable because the mutual action with the dyes is not enough to improve the orientation property.

The molecular weight of the saccharides is preferably 1,000 or less, and more preferably 700 or less. When the molecular weight of the saccharides is too large, a phase separation from the dye will occur, and it is not preferable because there is a fear of deteriorating the orientation property of the dye film.

The number of carbon atoms in the saccharides is usually 36 or less and preferably 24 or less. When the number of carbon atoms in the saccharides is too large, the molecular weight of the saccharides becomes so many that the phase separation from the dye will occur and it is not preferable because there is a fear of deteriorating the orientation property of the dye film.

Among the saccharides that can be used in the present invention, monosaccharide's, oligosaccharides, and monosaccharide alcohol are preferable because they satisfy the above-mentioned optimum number of hydroxy groups and the optimum range of the molecular weight.

Examples of the monosaccharide include xylose, ribose, glucose, fructose, mannose, sorbose, and galactose.

Examples of the oligosaccharide include trehalose, kojibiose, nigerose, maltose, maltotriose, isomaltotriose, maltotetraose, isomaltose, sophorose, laminaribiose, cellobiose, gentiobiose, lactose, sucrose, melibiose, tutinose, primeverose, turanose, panose, isopanose, cellotriose, manninotriose, solatriose, melezitose, planteose, gentianose, umbelliferose, raffinose, and stachyose.

Examples of the sugar alcohol include compounds made by reducing the above-mentioned monosaccharide's or oligosaccharides such as threitol, xylitol, ribitol, arabinitol, sorbitol, and mannitol.

Particularly preferable saccharides are xylose, mannose, maltose, maltotriose, and arabinitol.

Additionally, there are optical isomers respectively in these saccharides and sugar alcohol. However, the optical isomer may be used alone in the composition of the present invention; or both of the isomers may be contained into the composition of the present invention. Further, only one kind of saccharide may be used in the composition of the present invention, or two or more kinds of saccharides may be used in combination

It is preferable that the content of the saccharides in the composition of the present invention with respect to the dye is within the range of from 0.1 to 1 in weight ratio. The lower limit of the above content is more preferably 0.2, further preferably 0.3. The upper limit is more preferably 0.7, further preferably 0.6. When the content of the saccharide exceeds the upper limit, it is not preferable because there is a fear that an orientation degree of the association decreases. When the content is under the lower limit, it is also not preferable because there is a fear that the content is insufficient for increasing the association degree of the dye association.

### [Antifungal Agent, Antibacterial Agent and Sterilizer]

A chemical agent having at least any of the function among antifungal activity, antibacterial activity and sterilization activity may be added into the composition of the present invention. An addition of these additives enables to improve a storage stability of the composition.

The chemical agent having at least any of the function among the antifungal activity, the antibacterial activity and the sterilization activity in the present invention may be the one having at least any of antifungal capability of suppressing development/growth/breeding of mold, sterilization capability causing extinct of microorganism, and antibacterial capability of suppressing development/growth/breeding of microorganism; and ordinary antifungal agent, bactericide, and antibacterial agent can be used. However, it is preferable that they do not deteriorate optical performance of the anisotropic film prepared by using the composition of the present invention. Examples of the chemical agent having at least any of the function among antifungal activity, antibacterial activity and sterilization activity to be used in the present invention include phenolic series such as conventional 2,4,4′-trichloro-2′-hydroxydiphenyl, chloride series such as chlorine dioxide, iodine series such as iodine, and quaternary ammonium salt series such as benzalkonium chloride.

Further, the examples include Proxel BDN, Proxel BD20, Proxel GXL, Proxel LV, Proxel XL, Proxel XL2 and Proxel Ultra10 (manufactured by Avecia Ltd., trade names) as the chemical agent containing, as an effective component, 1,2-benzisothiazoline-3-one; Proxel IB (manufactured by Avecia Ltd, trade name) as the chemical agent containing, as an effective component, polyhexamethylene biguanide hydrochloride; and Densil P (manufactured by Avecia Ltd, trade name) as the chemical agent containing, as an effective component, dithio-2,2′-bis(benzmethylamide).

Furthermore, a compound represented by formula (II) is also effective and it is preferable in particular because it exhibits antibacterial activity effect with an ultratrace level.

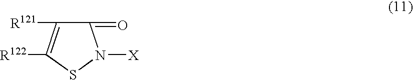

In formula (11), X represents a substituted or unsubstituted alkyl group, a substituted or unsubstituted cycloalkyl group or a substituted or unsubstituted aromatic hydrocarbon ring group; and R¹²¹ and R¹²² each independently represent a hydrogen atom, a halogen atom or an alkyl group.

The alkyl group represented by X is preferably an alkyl group having 1 to 6 carbon atoms, and more preferably an alkyl group having a substituent(s). Examples of the substituent of the alkyl group include a hydroxy group, a halogen atom, a cyano group, a phenylamino group, a halophenylamino group, a carboxy group, an alkoxycarbonyl group, an alkoxy group, an aryloxy group, a morpholino group, a piperidino group, a pyrrolidino group, a carbamoyloxy group or an isothiazolonyl group. Further, a chlorine atom and a bromine atom are preferable as the halogen atom and a halogen atom in the halophenyl group; a linear or branched-chain-shaped alkoxy group having 1 to 6 carbon atoms is preferable as the alkoxy group and an alkoxy group in the alkoxycarbonyl group; and an unsubstituted phenyl group and a phenyl group substituted by a lower alkyl group such as a methyl group, an ethyl group are preferable as the aryl group in the aryloxy group, respectively.

The cycloalkyl group represented by X is preferably a cycloalkyl group having 5 to 7 carbon atoms, and more preferably a cyclohexyl group. The substituent on the cycloalkyl group is preferably an alkyl group having 1 to 6 carbon atoms.

The aromatic hydrocarbon ring group represented by X is preferably a phenyl group. The aromatic hydrocarbon ring group preferably has a substituent(s). The substituent on the aromatic hydrocarbon ring group is preferably a nitro group, an alkyl group or an alkoxycarbonyl group. As the alkyl group, a lower alkyl group is preferable, and a methyl group and an ethyl group are particularly preferable. Further, an alkoxycarbonyl group having 2 to 7 carbon atoms is preferable as the above alkoxycarbonyl group.

Among those, it is preferable for the group represented by X to be an alkyl group having 1 to 6 carbon atoms which is substituted by a halogen atom, a hydroxy group, a cyano group or a morpholino group; to be a cycloalkyl group which may be substituted by an alkyl group having 1 to 6 carbon atoms; or an aromatic hydrocarbon ring group which is substituted by a halogen atom, a nitro group, an alkyl group having 1 to 6 carbon atoms.

R¹²¹ and R¹²² each independently represent a hydrogen atom, a halogen atom or an alkyl group. The halogen atom is preferably a chlorine atom or a bromine atom. The alkyl group is preferably an alkyl group having 1 to 6 carbon atoms. R¹²¹ is preferably a hydrogen atom or a halogen atom, and more preferably a hydrogen atom. R¹²² is preferably a halogen atom.

Additionally in the present invention, a phrase “which may be substituted” means that one or more substituent may be further substituted on the substituent. In addition, when the number of carbon atoms of the alkyl group or the alkyl moiety of a substituent is 3 or more, the component may be either linear or branched-chain-shaped.

Typical examples of the compound represented by formula (II) will be shown in the following.

No. Name of the Compound
1. 2-chloromethyl-5-chloro-3-isothiazolon
2. 2-cyanomethyl-5-chloro-3-isothiazolon
3. 2-hydroxymethyl-5-chloro-3-isothiazolon
4. 2-(3-methylcyclohexyl)-3-isothiazolon
5. 2-(4-chlorophenyl)-4,5-dichloro-3-isothiazolon
6. 2-(4-ethylphenyl)-3-isothiazolon
7. 2-(4-nitrophenyl)-5-chloro-3-isothiazolon
8. 2-chloromethyl-3-isothiazolon
9. 2-methoxyphenyl-4-methyl-5-chloro-3-isothiazolon
10. 2-morpholinomethyl-5-chloro-3-isothiazolon

Those compounds are possibly synthesized with reference to, for example, JP-A-2-278. Alternatively, commercially available marketing products such as Tribactran (trade name; manufactured by Hoechst AG) are also utilizable.

In addition, only a single chemical agent having at least any of the function among the antifungal activity, antibacterial activity and sterilization activity to be used in the present invention may be used, or two or more kinds of chemical agents may be used in combination.

Although the content of the chemical agent having at least any of the function among the antifungal, the antibacterial activity and the sterilization activity contained in the dichroic dye composition is not limited in particular, it is usually 0.01 mass % or more, preferably 0.001 mass % or more and on the other hand, usually 0.5 mass % or less and preferably 0.3 mass % or less. When the content of the chemical agent having at least any of the function among the antifungal activity, the antibacterial activity and the sterilization activity is too little, the dichroic dye composition does not have a sufficient antifungal effect, antibacterial effect or sterilization effect. When the content is too much, because the chemical agent precipitates among the dichroic dye composition, and there is a fear that a phase separation occurs when the anisotropic dye film is formed, there is an anxiety of causing optical defect such as a point defect, light scattering or so.

Because the light absorption anisotropic film of the present invention has a high dichroic ratio, it is preferable that the dichioic dye composition of the present invention contains Electron-Deficient disc-shape compound and Electron-Rich compound. In the present invention, for example, compounds disclosed in JP-A-2006-323377 are usable as the Electron-Deficient disc shape compound and Electron-Rich compound.

When the total mass of the composition is settled to be 100 mass parts, the ratio of the Electron-Deficient disc-shape compound in the composition of the present invention is usually 0.1 mass parts or more, preferably 0.2 mass parts or more, usually 50 mass parts or less, and preferably 40 mass parts or less. When the amount of the above compound is too little, there is a fear that any effect due to the use of Electron-Deficient disc-shape compound cannot be achieved. When the amount is too large, the viscosity of the composition as a solution becomes high, and it is not preferable because of its uneasy treating

When the total mass of the composition is settled to be 100 mass parts, the ratio of the Electron-rich compound in the composition of the present invention is usually 50 mass parts or less, preferably 40 mass parts or less. When the amount of the compound is too large, the viscosity of the composition as a solution becomes high, and it is not preferable because of its uneasy treating.

Further, it is preferable that mass fraction of Electron-Deficient disc-shape compound and Electron-Rich compound is usually within the range of from 10/90 to 90/10. When the mass fraction is not within the above range, it is not preferable because there is a fear that any effect due to the use of Electron-Deficient disc-shape compound or Electron-Rich compound cannot be achieved.

### [Solvent for Preparing a Coating Liquid]

It is preferable for the light absorption anisotropic film of the present invention to be formed by using the coating liquid containing the dichroic dye composition of the present invention. The solvent which is used for preparing the coating liquid is desirably selected from organic solvents. Examples of the organic solvent include amides such as N,N-dimethylformamide, sulfoxides such as dimethylsulfoxide, heterocyclic compounds such as pyridine, hydrocarbons such as benzene or hexane, alkyl halides such as chloroform or dichloromethane, esters such as methyl acetate or butyl acetate, ketones such as acetone or methylethyl ketone and ethers such as tetrahydrofliran or 1,2-dimethoxyethane. Among these, alkyl halides or ketones are preferred. Plural types of organic solvents may be used in combination.

### [Coating Manner]

The coating liquid may be applied by ordinary techniques (e.g., wire bar coating, extrusion coating, direct gravure coating, reverse gravure coating, die coating and inkjet method). The coating liquid preferably contains the dichroic dye composition in an amount from 1 to 20 mass % more preferably from 1 to 10 mass %, and further preferably from 1 to 5 mass %.

It is preferable for the light absorption anisotropic film of the present invention to be formed in accordance with a wet film-forming method. For the purpose of producing the light absorption anisotropic film in the present invention, after preparing the dichroic dye composition of the present invention, publicly known methods of applying the composition onto various substrates such as glass plate, so that the dye is orientated and laminated are adopted.

As the wet film-forming method, for example, a known method as disclosed in e.g. “Coating Engineering”, Yuji Harasaki (Asaku Shoten K. K., published on Mar. 20, 1971) pages 253-277 or “Creation and Applications of Harmonized Molecular Materials” supervised by Kunihiro Ichimura (CMC Publishing Co., Ltd., published on March. 3, 1998) pages 118-149, or a method of coating on a substrate preliminarily subjected to an alignment treatment by means of e.g. spin coating, spray coating, bar coating, roll coating, blade coating, free span coating, dye coating, or inkjet method may be mentioned.

The temperature at the time of coating is preferably from 0° C. to 80° C. Further, the humidity is preferably from 10% RH to 80% RH.

Because there may be a case where the light absorption anisotropic film produced by such a manner is inferior in its mechanical strength, a protective layer is provided on the film as occasion demand for its usage. The protective layer is formed by, for example, lamination of a transparent polymer film such as a triacetate, acryl, polyester, polyimide, triacetylcellulose or urethane type film and is used practically.

Further, in a case where the light absorption anisotropic film of the present invention is used as e.g. a polarizing filter for various display devices such as LCD or OWED, the anisotropic film may be formed directly on e.g. an electrode substrate constituting such a display device, or a substrate having the dye film formed thereon may be used as a constituting component of such a display device.

In the present invention, the light absorption anisotropic film is formed by applying the dichroic dye composition of the present invention on a support orientated unilaterally in the direction having an angle not parallel with respect to the orientation treatment direction. Further, it is more preferable that the dichroic dye composition of the present invention is applied in the direction almost the same as longitudinal or lateral direction of the support. By the above process, a light absorption anisotropic film without any optical defect and having high dichroic ratio can be provided. In addition, after applying the dichroic dye composition, cutting out the support for the purpose of providing a necessary polarization angle is not required, and accordingly, the productivity is high.

JP-A-2007-127987, for example, discloses about preferred coating manners for the dichroic dye composition of the present invention.

In the present invention, it is possible after applying the dichroic dye composition coating liquid onto the surface of the alignment film and forming a light absorption anisotropic film, to allow the organic solvent evaporating by reducing pressure, and to dry the light absorption anisotropic film. Accordingly, the light absorption anisotropic film having high dichroic ratio can be provided.

In this occasion, reducing pressure means that the light absorption anisotropic film is left under the condition of reduced pressure. At this moment, it is preferable that the support having the light absorption anisotropic film is maintained to be horizontal without moving from the higher position toward the lower position.

Regarding with the time interval before starting the pressure reduction treatment of the light absorption anisotropic film after coating, the shorter, the better, and it is preferable to be from 1 second to 30 seconds.

Examples of the method for pressure reducing treatment include the following methods. Namely, the light absorption anisotropic film prepared by applying the coating liquid onto the support is introduced into a pressure reducing apparatus and receive the pressure reduction treatment. For example, the pressure reduction apparatus illustrated in FIG. 9 or FIG. 10 of JP-A-2006-201759 can be used. JP-A-2004-169975 discloses about the pressure reducing apparatus in detail.

With regard to the condition of pressure reducing treatment, the pressure among the system in which the dye film exists is preferably 2×10⁴ Pa or less, further preferably 1×10⁴ Pa or less and particularly preferably 1×10³ Pa or less. In addition, it is preferably 1 Pa or more, and further preferably 1×10¹ Pa or more. Usually, it is preferable for the pressure to which the system reaches finally to be as the above description. When the pressure is too high, there is a fear that the drying becomes impossible and orientation is disturbed. When the pressure is too low, the drying becomes so rapid that there is a fear of generating defects.

Further, the time for pressure reduction treatment is preferably from 5 seconds to 180 seconds. When the time is too long, there is a fear that the rapid drying of the dye film before relaxation of the orientation becomes impossible and the orientation is disturbed. When the time is too short, there is a fear that the drying becomes impossible and the orientation is disturbed.

Further, with regard to the temperature among the system in the occasion of the pressure reducing treatment, it is preferably from 10° C. to 60° C. When the temperature is too high, there is a fear that convection occurs during the drying and nonuniformity generates in the coated film. When the temperature is too low, there is a fear that the drying becomes impossible and the orientation is disturbed.

Further, when the dye film is applied by the wet process film forming method, the support may be warmed or may be cooled too. Further, the temperature of the support in this occasion is preferably from 10° C. to 60° C. When the temperature is too high, there is a fear that the orientation is disturbed before being dried under reduced pressure. When the temperature is too low, there is a fear that water drop attaches onto the support and obstructs the coating. When the dye film coated in accordance with the wet process film forming method is dried under the reduced pressure, the support may be warmed. The temperature of the support in this occasion is preferably 60° C. or less. When the temperature is too high, there is a fear that the orientation is disturbed before being dried under reduced pressure.

### [Properties of Light Absorption Anisotropic Layer]

The light absorption anisotropic layer has a thickness of preferably 0.01 to 2 m, more preferably of 0.05 to 1 μm, and further preferably of 0.05 to 0.5 μm.

When the coating liquid of the dichroic dye composition of the present invention is applied to a surface of the alignment film, the dichroic dye may be aligned with a tilt angle of an alignment film at an alignment layer interface and with a tilt angle of air interface at an air interface. After applying the coating liquid of the dichroic dye composition of the present invention to the surface of the alignment film, the dichroic dye is aligned uniformly, to achieve horizon alignment state.

The light absorption anisotropic film formed by horizontally orientating the dichroic dye and by fixing the oriented state can be used as a polarizing element.

The dichroic dye composition of the present invention preferably contains a horizontally orientating agent. The horizontally orientating agent that can be preferably used in the present invention is preferably a fluoro-aliphatic group-containing compound represented by formula (III) or a polymer comprising at least one kind of polymerization unit of a fluoro-aliphatic group-containing monomer represented by formula (IV) or (V) and at least one kind of polymerization unit of an amide group-containing monomer represented by formula (VI).

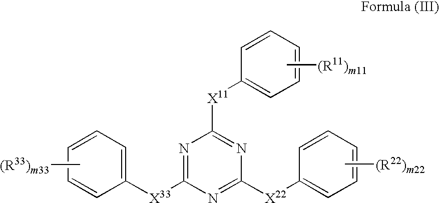

In formula (III), R¹¹, R¹² and R¹³ each independently represent an alkoxy group having CF₃ group(s) or CF₂H group(s) at its terminal; X¹¹, X²² and X³³ each independently represent —NH—, —O— or —S—; and m¹¹, m²² and m³³ each independently represent an integer of 1 to 3.

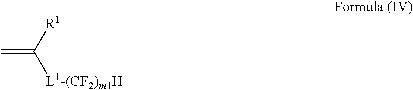

In formula (IV), R¹ represents a hydrogen atom, a halogen atom or a methyl group; L¹ represents a divalent linking group; and m1 represents an integer of 1 to 18.

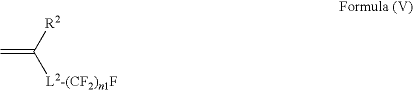

In formula (V), R² represents a hydrogen atom, a halogen atom or a methyl group; L² represents a divalent linking group; and n1 represents an integer of 1 to 18.

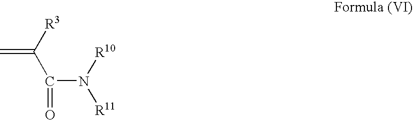

In formula (VI), R³ represents a hydrogen atom, a halogen atom or a methyl group; R¹⁰ and R¹¹ each independently represent a hydrogen atom, an alkyl group having 1 to 18 carbon atoms, an aromatic group having 6 to 20 carbon atoms, or a heterocyclic group having 1 to 20 carbon atoms; and R¹⁰ and R¹¹ may be bonded with each other to form a heterocyclic group.

First, the compound represented by formula (III) is explained.

R¹¹, R²² and R³³ each independently represent an alkoxy group having CF₃ group(s) or CF₂H group(s) at its terminal. The alkoxy group may be a straight chain form or a branched chain form. The number of carbon atoms in the alkoxy group is preferably from 4 to 20, more preferably from 4 to 16, and particularly preferably from 6 to 16. The alkoxy group having CF₃ group(s) or CF₂H group(s) at its terminal is an alkoxy group in which hydrogen atoms are partially or entirely replaced by fluorine atoms. In this case, the hydrogen atoms in the alkoxy group are replaced by fluorine atoms in a ratio of preferably 50% or more, more preferably 60% or more, and particularly preferably 70% or more. Specific examples of the alkoxy group having CF₃ group(s) or CF₂H group(s) at its terminal represented by R¹¹, R²² and R³³ are shown below, but the present invention is not limited to these.

R1: n-C₈F₁₇—O—
R2: n-C₆F₁₃—O—
R3: n-C₄F₉—O—
R4: n-C₉F₁₇-(CH₂)₂—O—(CH₂)₂—O—
R5: n-C₆F₁₃—(CH₂)₂—O—(CH₂)₂—O—
R6: n-C₄F₉—(CH₂)₂—O—(CH₂)₂—O—
R7: n-C₈F₁₇—(CH₂)₃—O—
R8: n-C₆F₁₃—(CH₂)₃—O—
R9: n-C₄F₉—(CH₂)₃—O—

### R10: H—(CF₂)₈—O—

### R11: H—(CF₂)₆—O—

### R12: H—(CF₂)₄—O—

### R13: H—(CF₂)₈—(CH₁₂)—O—

### R14: H—(CF₂)₆(CH₂)—O—

### R15: H—(CF₂)₄—(CH₂)—O—

### R16: H—(CF₂)₈—(CH₂)—O—(CH₂)₂—O—

### R17: H—(CF₂)₆—(CH₂)—O—(CH₂)₂—O—

### R18: H—(CF₂)₄—(CH₂)—O—(CH₂)₂—O—

In formula (III), X¹¹, X²² and X³³ each preferably represent —NH— or —O—; more preferably —NH—. m¹¹, m²² and m³³ each are preferably 2.

The specific examples of the compound represented by formula (III) are shown below. However, the present invention is not limited to these specific examples.

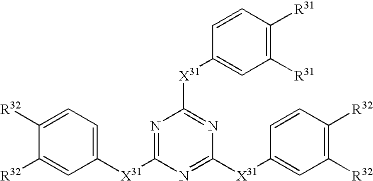

| Compound | | | |
|---|---|---|---|
| No. | R³¹ | R³² | X³¹ |
| I-1 | O(CH₂)₃(CF₂)₄F | O(CH₂)₃(CF₂)₄F | NH |
| I-2 | O(CH₂)₃(CF₂)₆F | O(CH₂)₃(CF₂)₆F | NH |
| I-3 | O(CH₂)₃(CF₂)₈F | O(CH₂)₃(CF₂)₈F | NH |
| I-4 | OCH₂(CF₂)₆H | OCH₂(CF₂)₆H | NH |
| I-5 | OCH₂(CF₂)₈H | OCH₂(CF₂)₈H | NH |
| I-6 | O(CH₂)₂O(CH₂)₂(CF₂)₆F | O(CH₂)₂O(CH₂)₂(CF₂)₆F | NH |
| I-7 | O(CH₂)₂O(CH₂)₂(CF₂)₄F | O(CH₂)₂O(CH₂)₂(CF₂)₄F | NH |
| I-8 | O(CH₂)₃S(CH₂)₂(CF₂)₆F | O(CH₂)₃S(CH₂)₂(CF₂)₆F | NH |
| I-9 | O(CH₂)₃S(CH₂)₂(CF₂)₄F | O(CH₂)₃S(CH₂)₂(CF₂)₄F | NH |
| I-10 | O(CH₂)₆S(CH₂)₂(CF₂)₆F | O(CH₂)₆S(CH₂)₂(CF₂)₆F | NH |
| I-11 | O(CH₂)₆S(CH₂)₂(CF₂)₄F | O(CH₂)₆S(CH₂)₂(CF₂)₄F | NH |
| I-17 | O(CH₂)₂O(CH₂)(CF₂)₆F | O(CH₂)₂O(CH₂)(CF₂)₆F | NH |
| I-18 | O(CH₂)₃(CF₂)₆F | O(CH₂)₃(CF₂)₆F | O |
| I-19 | OCH₂(CF₂)₆H | OCH₂(CF₂)₆H | O |
| I-20 | O(CH₂)₂O(CH₂)₂(CF₂)₆F | O(CH₂)₂O(CH₂)₂(CF₂)₆F | O |
| I-21 | O(CH₂)₃S(CH₂)₂(CF₂)₆F | O(CH₂)₃S(CH₂)₂(CF₂)₆F | O |
| I-22 | O(CH₂)₂O(CH₂)(CF₂)₆H | O(CH₂)₂O(CH₂)(CF₂)₆H | O |
| I-23 | O(CH₂)₃(CF₂)₆F | O(CH₂)₃(CF₂)₆F | S |
| I-24 | OCH₂(CF₂)₆H | OCH₂(CF₂)₆H | S |
| I-25 | O(CH₂)₂O(CH₂)₂(CF₂)₆F | O(CH₂)₂O(CH₂)₂(CF₂)₆F | S |
| I-26 | O(CH₂)₃S(CH₂)₂(CF₂)₆F | O(CH₂)₃S(CH₂)₂(CF₂)₆F | S |
| I-27 | O(CH₂)₂O(CH₂)(CF₂)₆H | O(CH₂)₃O(CH₂)(CF₂)₆H | S |

The compound having a 1,3,5-triazine ring represented by formula (III) can be easily synthesized referring to the method described in, for example, JP-A-2002-20363.

Synthesis examples of the compound represented by formula (III) will be explained firstly.

### [Synthesis of Compound No. (I-6)]

The compound No. (I-6) can be synthesized according to the following scheme. In the following scheme, Bu represents a butyl group.

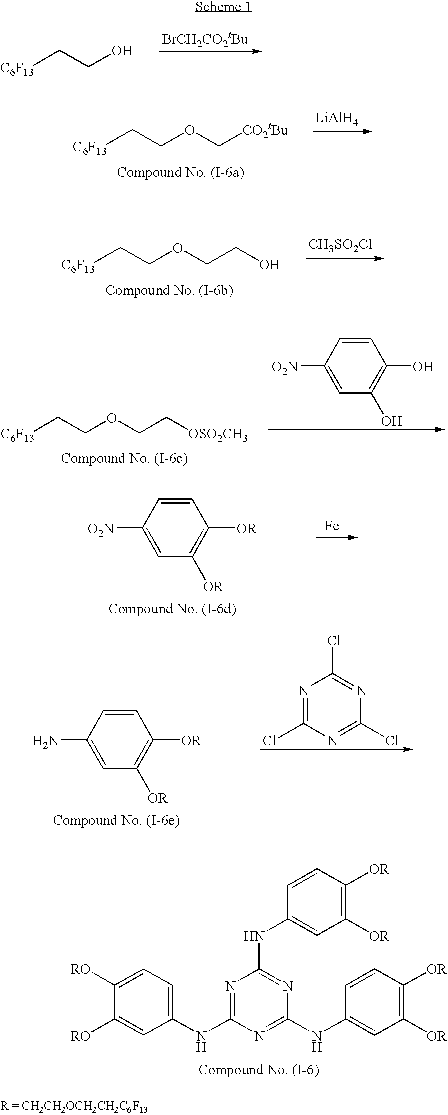

### Synthesis of Compound No. (I-6a)

After dissolving 2-(1,1,2,2-tetrahydrotridecafluoro octyloxy)ethanol (36.4 g, 0.10 mol) and tetrabutylammonium bisulfate (6.8 g, 0.020 mol) into tetrahydrofuran (50 mL), 50% sodium hydroxide aqueous solution (50 mL) and tert-butyl bromoacetate (29.3 g, 0.15 mol) were sequentially added thereto dropwise at 0° C., and the resultant mixture was stirred at 20° C. or lower for 6 hours. Adding ethyl acetate (100 mL) into the reaction liquid and after extracting with 2 mol/l of hydrochloric acid water, evaporation was conducted to obtain 45.4 g of an aimed substance being a yellow oily substance. This corresponds to 95% of theoretical yield.

¹H-NMR (CDCl₃): δ 4.0 (s, 2H), 3.8 (t, 2H), 2.5 (m, 2H), 1.5 (s, 9H)

Synthesis of Compound No. (I-6b)

Under nitrogen atmosphere, after suspending lithium hydride aluminum (4.0 g, 0.11 mol) into tetrahydrofuran (150 mL), Compound No. (I-6a) (47.8 g, 0.10 mol) was added dropwise at 0° C. and the resultant mixture solution was stirred at the room temperature for 2 hours. Adding 3 mol/l of sodium hydroxide aqueous solution (140 mL) and ethyl acetate (300 mL) sequentially into the reaction liquid, and after filtrating through celite, evaporation of the organic layer was conducted to obtain 34.9 g of an aimed substance being an achromatic transparent oily substance. This corresponds to 86% of theoretical yield.

¹H-NMR (CDCl₃): δ 3.7-3.9 (m, 4H), 3.6 (t, 2H), 2.5 (m, 2H)

### Synthesis of Compound No. (I-6c)

After dissolving Compound No. (I-6b) (34.9 g, 0.086 mol) and triethylamine (14.5 mL, 0.10 mol) into ethyl acetate (200 mL), methanesulfonyl chloride (11.8 g, 0.10 mol) was added dropwise at 0° C. and the resultant mixture solution was stirred at the room temperature for 2 hours. Adding ethyl acetate (100 mL) into the reaction liquid and after extracting with saturated sodium chloride solution, evaporation was conducted to obtain 41.6 g of an aimed substance being a yellow oily substance. This corresponds to 99% of theoretical yield.

¹H-NMR (CDCl₃) δ 4.4 (t, 2H), 3.7-3.9 (m, 4H), 3.0 (s, 3H), 2.5 (m, 2H)

### Synthesis of Compound No. (I-6d)

After suspending Compound No. (I-6c) (48.6 g, 0.10 mol), 4-nitrocatechol (7.1 g, 0.045 mol) and potassium carbonate (20.1 g, 0.15 mol) into dimethylacetamide (150 mL), the resultant suspended solution was stirred at 110° C. for 3 hours. Adding ethyl acetate (100 mL) into the reaction liquid and after extracting with saturated sodium chloride solution, evaporation was conducted to obtain 41.4 g of an aimed substance being a yellow powder. This corresponds to 95% of theoretical yield.

¹H-NMR (CDCl₃): δ 7.9 (dd, 1H), 7.8 (d, 1H), 6.9 (d, 1H), 4.2 (t, 4H), 3.7-3.9 (m, 8H), 2.5 (m, 4H)

### Synthesis of Compound No. (I-6e)

Suspending reduced iron (24 g, 0.43 mol) and ammonium chloride (0.78 g, 0.014 mol) into a mixed solvent of 2-propanol (450 mL) and water (90 mL), and after refluxing it under heating for 30 minutes, Compound No. (I-6d) (40 g, 0.043 mol) was dividedly added, followed by further refluxing for 5 hours. After filtrating the reaction liquid through celite, evaporation was conducted to obtain 36.8 g of an aimed substance being a yellow oily substance. This corresponds to 95% of theoretical yield.

¹H-NMR (CDCl₃): δ 6.8 (d, 1H), 6.3 (s, 1H), 6.2 (d, 1H), 4.1 (m, 4H), 3.7-3.9 (m, 8H), 2.5 (m, 4H)

### Synthesis of Compound No. (I-6)

After suspending Compound No. (I-6e) (36.8 g, 0.041 mol) and cyanuric chloride (2.27 g, 0.012 mol) into methylethylketone (150 mL), potassium carbonate (5.67 g, 0.041 mol) was added and the resultant suspension was refluxed under heating for 3 hours. After adding water (400 mL) into the reaction liquid, white precipitates were taken out by filtration, and re-crystallizing with hexane, 28.8 g of an aimed substance was obtained. This corresponds to 86% of theoretical yield.

¹H-NMR (CDCl₃): δ 7.2 (br, 3H), 7.1 (br, 3H), 6.9 (br, 3H), 6.8 (d, 3H), 3.9-4.2 (m, 12H), 3.6-3.8 (m, 24H), 2.5 (m, 12H)

Next, the compounds represented by formula (IV) or (V) are explained.

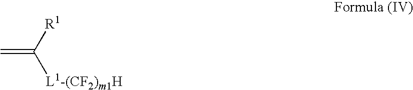

In formula (IV), R¹ represents a hydrogen atom, a halogen atom or a methyl group. R¹ is preferably a hydrogen atom or a methyl group. L¹, represents a divalent linking group. m1 represents an integer of 1 to 18. m1 is preferably an integer of 2 to 12, more preferably 4 to 8, and most preferably 4 or 6.

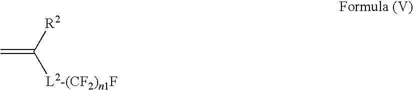

In formula (V), R² represents a hydrogen atom, a halogen atom or a methyl group. R² is preferably a hydrogen atom or a methyl group. L² represents a divalent linking group. n¹ represents an integer of 1 to 18. n1 is preferably an integer of 2 to 12, more preferably 4 to 8, and most preferably 4 or 6.

The divalent linking group represented by L1 and L2 will be described below. Although L¹ and L² are not restricted as far as they each independently represents a divalent substituent, it is preferable that they have a structure represented by formula (VII). In formula (VII), (a) illustrates a bonding position at the double bond side, and (b) illustrate a bonding position at the fluoro-aliphatic group side respectively.

(a)-X¹⁰—R²⁰—(b) Formula (VII)

In formula (VII), X¹⁰ represents a single bond or a divalent linking group expressed by *—COO—**, *—COS—**, *—OCO—**, *—CON(R²¹)—**, or *—O—**. In this occasion, * illustrates a bonding position the double bond side, and ** illustrates a bonding position at R²⁰.

R²⁰ represents a polymethylene group (for example, a methylene group, an ethylene group, or a trimethylene group) which may have a substituent, a phenylene group (for example, o-phenylene group, m-phenylene group, p-phenylene group) which may have a substituent, and a group which can be arbitrarily formed by combination of those. Among those, the polymethylene group is more preferable; the methylene group, the ethylene group, the trimethylene group and the tetramethylene group are preferable among the polymethylene group, and the methylene group and the ethylene group are further preferable.

R²¹ represents a hydrogen atom, a substituted or unsubstituted alkyl group having 1 to 8 carbon atoms, or a substituted or unsubstituted aryl group having 6 to 20 carbon atoms. R²¹ is preferably a hydrogen atom or an alkyl group having 1 to 6 carbon atoms, more preferably a hydrogen atom or an alkyl group having 1 to 4 carbon atoms.

The fluoro-aliphatic group-containing monomer represented by formula (IV) is more preferably a monomer represented by formula (VIII).

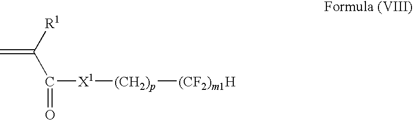

In formula (VIII), X¹ represents a divalent linking group selected from —O—, —S— and —N(R²²²)—. p represents an integer of 1 to 8. X¹ is preferably —O— or —N(R²²²)—, more preferably —O—. p is preferably an integer of 1 to 6, more preferably an integer of 1 to 3. R¹ and m1 have the same meanings as those in the formula (IV), and the preferable ranges thereof are also the same as those in the formula (IV). R²²² represents a hydrogen atom, a substituted or unsubstituted alkyl group having 1 to 8 carbon atoms, or a substituted or unsubstituted aryl group having 6 to 20 carbon atoms.

The fluoro-aliphatic group-containing monomer represented by formula (V) is more preferably a monomer represented by formula (IX).

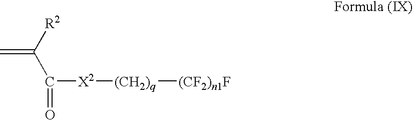

In formula (IX), X² represents a divalent linking group selected from —O—, —S— and —N(R²²²)—. q represents an integer of 1 to 8. X² is preferably —O— or —N(R²²²)—, more preferably —O—. q is preferably an integer of 1 to 6, more preferably an integer of 1 to 3. R² and n1 have the same meanings as those in the formula (V), and the preferable ranges thereof are also the same as those in the formula (V). R²²² has the same meaning as that in the formula (VIII).

The specific examples of the fluoro-aliphatic group-containing monomer represented by formula (IV) are shown below. However, the present invention is not limited to these specific examples. Herein, Ph represents a phenyl group.

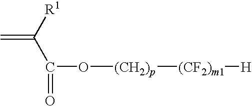

| | R¹ | p | m¹ |
|---|---|---|---|
| F-1 | H | 1 | 4 |
| F-2 | CH₃ | 1 | 4 |
| F-3 | F | 1 | 4 |
| F-4 | H | 2 | 4 |
| F-5 | CH₃ | 3 | 4 |
| F-6 | H | 1 | 6 |
| F-7 | CH₃ | 1 | 6 |
| F-8 | F | 1 | 6 |
| F-9 | H | 2 | 6 |
| F-10 | CH₃ | 2 | 6 |
| F-11 | H | 3 | 6 |
| F-12 | H | 1 | 8 |
| F-13 | CH₃ | 1 | 8 |
| F-14 | F | 1 | 8 |
| F-15 | CH₃ | 2 | 8 |
| F-16 | H | 3 | 8 |
| F-17 | CH₃ | 3 | 8 |
| F-18 | H | 1 | 10 |
| F-19 | CH₃ | 1 | 10 |
| F-20 | F | 1 | 10 |
| F-21 | H | 2 | 10 |
| F-22 | H | 2 | 10 |
| F-23 | H | 1 | 12 |
| F-24 | CH₃ | 1 | 12 |
| F-25 | F | 1 | 12 |
| F-26 | H | 2 | 12 |
| F-27 | H | 3 | 12 |

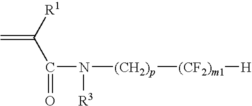

| | R¹ | R³ | p | m¹ |
|---|---|---|---|---|
| F-28 | H | H | 1 | 4 |
| F-29 | CH₃ | H | 1 | 4 |
| F-30 | H | CH₃ | 1 | 4 |
| F-31 | H | H | 2 | 4 |
| F-32 | H | H | 1 | 6 |
| F-33 | CH₃ | H | 1 | 6 |
| F-34 | H | CH₃ | 1 | 6 |
| F-35 | H | C₂H₅ | 1 | 6 |
| F-36 | CH₃ | H | 1 | 6 |
| F-37 | F | H | 2 | 6 |
| F-38 | H | H | 1 | 8 |
| F-39 | CH₃ | H | 1 | 8 |
| F-40 | H | CH₃ | 1 | 8 |
| F-41 | H | CH₄H₉ (n) | 1 | 8 |
| F-42 | CH₃ | C₂H₅ | 1 | 8 |
| F-43 | H | CH₂Ph | 1 | 8 |
| F-44 | H | H | 2 | 8 |
| F-45 | CH₃ | H | 3 | 8 |
| F-46 | H | H | 1 | 10 |
| F-47 | CH₃ | CH₃ | 1 | 10 |
| F-48 | H | H | 1 | 12 |
| F-49 | CH₃ | H | 1 | 12 |

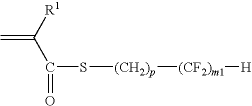

| | R¹ | p | m¹ |
|---|---|---|---|
| F-50 | H | 1 | 4 |
| F-51 | CH₃ | 1 | 4 |
| F-52 | H | 2 | 4 |
| F-53 | H | 1 | 6 |
| F-54 | CH₃ | 1 | 6 |
| F-55 | CH₃ | 2 | 6 |
| F-56 | H | 1 | 8 |
| F-57 | CH₃ | 1 | 8 |
| F-58 | F | 1 | 8 |
| F-59 | H | 2 | 8 |
| F-60 | CH₃ | 3 | 8 |
| F-61 | H | 1 | 10 |
| F-62 | CH₃ | 1 | 10 |
| F-63 | H | 1 | 12 |
| F-64 | CH₃ | 1 | 12 |

| | |
|---|---|
| | F-65 |
| 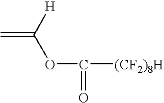 | |
| | F-66 |
| 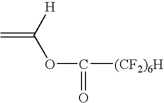 | |
| | F-67 |
| 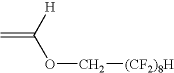 | |
| | F-68 |
| 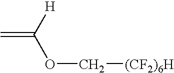 | |
| | F-69 |
| 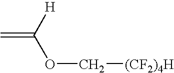 | |
| | F-70 |
| 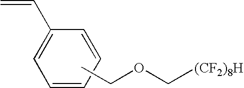 | |
| | F-71 |
| 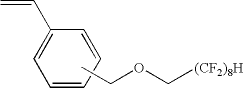 | |
| | F-72 |
| 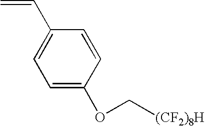 | |
| | F-73 |
| 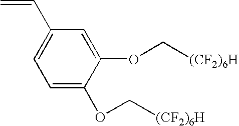 | |
| | F-74 |
| 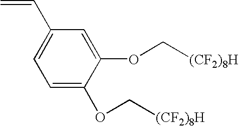 | |

The specific examples of the fluoro-aliphatic group-containing monomer represented by formula (V) are shown below. However, the present invention is not limited to these specific examples. Herein, Ph represents a phenyl group.

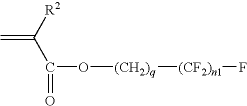

| | R² | q | n¹ |
|---|---|---|---|
| F-75 | H | 1 | 2 |
| F-76 | CH₃ | 1 | 2 |
| F-77 | F | 1 | 2 |
| F-78 | H | 6 | 2 |
| F-79 | CH₃ | 6 | 2 |
| F-80 | H | 1 | 3 |
| F-81 | CH₃ | 1 | 3 |
| F-82 | F | 1 | 3 |
| F-83 | H | 2 | 4 |
| F-84 | CH₃ | 2 | 4 |
| F-85 | F | 2 | 4 |
| F-86 | H | 3 | 4 |
| F-87 | CH₃ | 3 | 4 |
| F-88 | H | 2 | 6 |
| F-89 | CH₃ | 2 | 6 |
| F-90 | F | 2 | 6 |
| F-91 | H | 3 | 6 |
| F-92 | CH₃ | 3 | 6 |
| F-93 | H | 6 | 6 |
| F-94 | H | 2 | 8 |
| F-95 | CH₃ | 2 | 8 |
| F-96 | F | 2 | 8 |
| F-97 | CH₃ | 3 | 8 |
| F-98 | H | 3 | 8 |
| F-99 | CH₃ | 6 | 8 |
| F-100 | H | 2 | 10 |
| F-101 | CH₃ | 2 | 10 |
| F-102 | F | 2 | 10 |

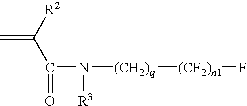

| | R² | R⁹ | q | n¹ |
|---|---|---|---|---|
| F-103 | H | H | 1 | 3 |
| F-104 | CH₃ | H | 1 | 3 |
| F-105 | H | CH₃ | 1 | 3 |
| F-106 | H | H | 2 | 3 |
| F-107 | H | H | 1 | 6 |
| F-108 | CH₃ | H | 1 | 6 |
| F-109 | H | CH₃ | 1 | 6 |
| F-110 | H | C₂H₅ | 1 | 6 |
| F-111 | CH₃ | H | 1 | 6 |
| F-112 | H | H | 2 | 6 |
| F-113 | H | H | 1 | 7 |
| F-114 | CH₃ | H | 1 | 7 |
| F-115 | H | CH₃ | 1 | 7 |
| F-116 | H | C₄H₉ (n) | 1 | 7 |
| F-117 | CH₃ | H | 1 | 7 |
| F-118 | H | H | 2 | 7 |
| F-119 | H | H | 1 | 8 |
| F-120 | CH₃ | H | 1 | 8 |
| F-121 | H | CH₃ | 1 | 8 |
| F-122 | H | C₄H₉ (n) | 1 | 8 |
| F-123 | CH₃ | C₂H₅ | 1 | 8 |
| F-124 | H | CH₂Ph | 1 | 8 |
| F-125 | H | H | 2 | 8 |
| F-126 | CH₃ | H | 3 | 8 |
| F-127 | H | H | 1 | 10 |
| F-128 | CH₃ | CH₃ | 1 | 10 |

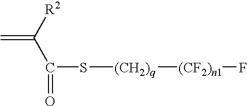

| | R² | q | n¹ |
|---|---|---|---|
| F-129 | H | 2 | 4 |
| F-130 | CH₃ | 2 | 4 |
| F-131 | H | 2 | 6 |
| F-132 | CH₃ | 2 | 6 |
| F-133 | H | 2 | 8 |
| F-134 | CH₃ | 2 | 8 |
| F-135 | H | 2 | 10 |
| F-136 | CH₃ | 2 | 10 |

| | |
|---|---|
| | F-137 |
| 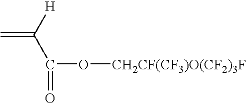 | |
| | F-138 |
| 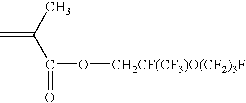 | |
| | F-139 |
| 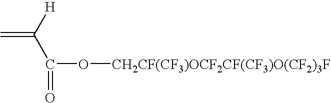 | |
| | F-140 |
| 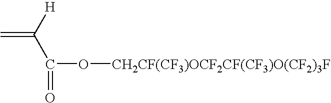 | |
| | F-141 |
| 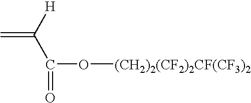 | |
| | F-142 |
| 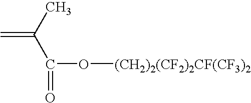 | |
| | F-143 |
| 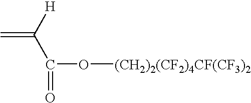 | |
| | F-144 |
| 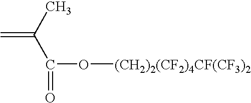 | |
| | F-145 |
| 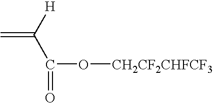 | |
| | F-146 |
| 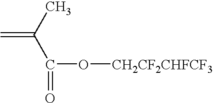 | |
| | F-147 |
| 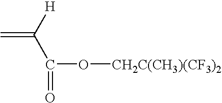 | |
| | F-148 |
| 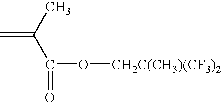 | |
| | F-149 |
| 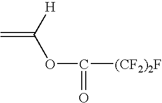 | |
| | F-150 |
| 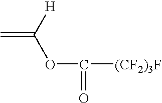 | |
| | F-151 |
| 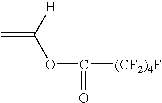 | |
| | F-152 |
| 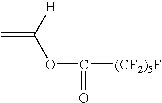 | |
| | F-153 |
| 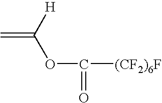 | |
| | F-154 |
| 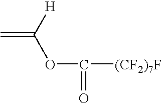 | |
| | F-155 |
| 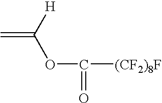 | |
| | F-156 |
| 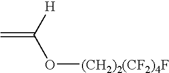 | |
| | F-157 |
| 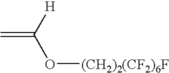 | |
| | F-158 |
| 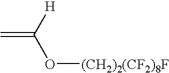 | |
| | F-159 |
| 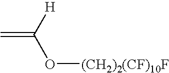 | |
| | F-160 |
| 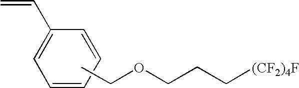 | |
| | F-161 |
| 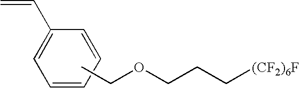 | |
| | F-162 |
| 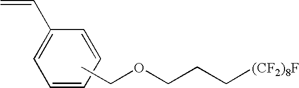 | |
| | F-163 |
| 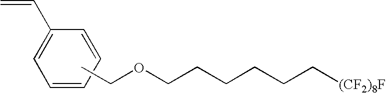 | |
| | F-164 |
| 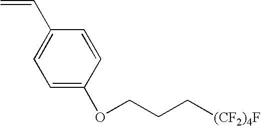 | |
| | F-165 |
| 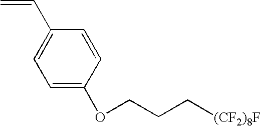 | |

The amide group-containing monomer that can be preferably used in the present invention will be described below. The structure of the amide group-containing monomer is not restricted as far as it contains the amide group, and it may contain any of the primary, secondary and tertiary amide group. In addition, the amide group may directly bond to the polymer principal chain, or may exist apart from it.

Preferred example of the polymer containing a fluorine atom being employable to the present invention is a polymer including polymerization unit of the amide group-containing monomer represented by formula (VI).

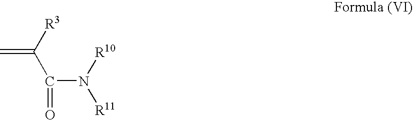

In formula (VI), R³ represents a hydrogen atom, a halogen atom or a methyl group. R³ is preferably a hydrogen atom or a methyl group. R¹⁰ and R¹¹ each independently represent a hydrogen atom, an alkyl group having 1 to 18 carbon atoms, an aromatic group having 6 to 20 carbon atoms, or a heterocyclic group having 1 to 20 carbon atoms. These substituents may be further substituted with a substituent. R¹⁰ and R¹¹ each are preferably an alkyl group having 1 to 12 carbon atoms, or an aromatic group having 6 to 15 carbon atoms; and further preferably an alkyl group having 1 to 6 carbon atoms, or an aromatic group having 6 to 12 carbon atoms. R¹⁰ and R¹¹ may be bonded with each other to form a heterocyclic group. Examples of the heterocycle to be formed include a pyrrolidine ring, a piperidine ring and a morpholine ring.

The specific examples of the amide group-containing monomer represented by formula (VI) that can be preferably used in the present invention are shown below. However, the present invention is not limited to these specific examples.

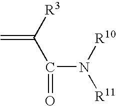

| | R³ | R¹⁰ | R¹¹ |
|---|---|---|---|
| A1 | H | CH₃ | CH₃ |
| A2 | H | C₂H₅ | C₂H₅ |
| A3 | H | C₄H₉ | C₄H₉ |
| A4 | H | C₆H₁₃ | C₆H₁₃ |
| A5 | H | CH₂CH₂OH | CH₂CH₂OH |
| A6 | H | CH₂CH₂OCH₃ | CH₂CH₂OCH₃ |
| A7 | H | H | CH(CH₃)₂ |
| A8 | H | H | CH₂CH(C₂H₅)C₄H₉ (n) |
| A9 | H | H | C(CH₃)₂CH₂COCH₃ |
| A10 | H | H | CH₃CH₂Ph |
| A11 | CH₃ | CH₃ | CH₃ |
| A12 | CH₃ | C₂H₅ | C₂H₅ |
| A13 | CH₃ | C₄H₉ | C₄H₉ |
| A14 | CH₃ | C₆H₁₃ | C₆H₁₃ |
| A15 | CH₃ | CH₂CH₂OH | CH₂CH₂OH |
| A16 | CH₃ | CH₂CH₂OCH₃ | CH₂CH₂OCH₃ |
| A17 | CH₃ | H | CH(CH₃)₂ |
| A18 | CH₃ | H | CH₂CH(C₂H₅)C₄H₉ (n) |
| A19 | CH₃ | H | C(CH₃)₂CH₂COCH₃ |
| A20 | CH₃ | H | CH₃CH₂Ph |
| A21 | H | H | Ph |
| A22 | H | CH₃ | Ph |
| A23 | H | Ph | Ph |
| A24 | H | H | 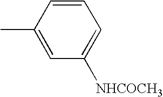 |
| A25 | H | H | Ph |
| A26 | H | CH₃ | Ph |
| A27 | H | Ph | Ph |
| A28 | H | H | 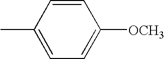 |

| | Structure |
|---|---|
| A29 | 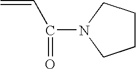 |
| A30 | 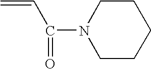 |
| A31 | 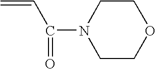 |
| A32 | 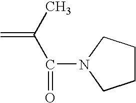 |
| A33 | 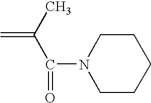 |
| A34 | 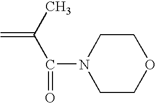 |
| A35 | 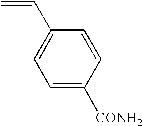 |
| A36 | 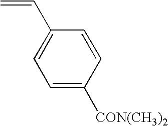 |
| A37 | 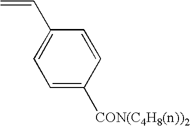 |
| A38 | 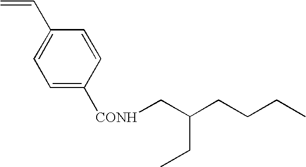 |

Examples of a substituent which may be possessed by the monomer represented by formula (IV), (V), (VI), (VIII) or (IX) include the following. The examples of the substituent include a hydroxy group, a halogen atom (such as Cl, Br, F and I), a cyano group, a nitro group, a carboxyl group, a sulfo group, a linear or cyclic alkyl group having 1 to 8 carbon atoms (such as methyl, ethyl, isopropyl, n-butyl, n-hexyl, cyclopropyl cyclohexyl, 2-hydroxyethyl, 4-carboxybutyl, 2-methoxyethyl, 2-diethylaminoethyl), an alkenyl group having 1 to 8 carbon atoms (such as vinyl, allyl, 2-hexenyl), an alkynyl group having 2 to 8 carbon atoms (such as ethynyl, 1-butynyl, 3-hexynyl), an aralkyl group having 7 to 12 carbon atoms (such as benzyl, phenethyl), an aryl group having 6 to 10 carbon atoms (such as phenyl, naphthyl, 4-carboxyphenyl, 4-acetoamidephenyl, 3-methanesulfoneamidephenyl, 4-methoxyphenyl, 3-carboxyphenyl, 3,5-dicarboxyphenyl, 4-methanesulphoneamidephenyl, 4-butanesulfoneamidephenyl), an acyl group having 1 to 10 carbon atoms (such as acetyl, benzoyl, propanoyl, butanoyl), an alkoxycarbonyl group having 2 to 10 carbon atoms (such as methoxycarbonyl, ethoxycarbonyl), an aryloxycarbonyl group having 7 to 12 carbon atoms (such as phenoxycarbonyl, naphtoxycarbonyl), a carbamoyl group having 1 to 10 carbon atoms (such as unsubstituted carbamoyl, methylcarbainoyl, diethylcarbamoyl, phenylcarbamoyl), an alkoxy group having 1 to 8 carbon atoms (such as methoxy, ethoxy, butoxy, methoxyethoxy), an aryloxy carbonyl group having 6 to 12 carbon atoms (such as phenoxy, 4-carboxyphenoxy, 3-methylphenoxy, naphtoxy), an acyloxy group having 2 to 12 carbon atoms (such as acetoxy, benzoyloxy), a sulfonyloxy group having 1 to 12 carbon atoms (such as methyl sulfonyloxy, phenyl sulfonyloxy), an amino group having 0 to 10 carbon atoms (such as unsubstituted amino, dimethylamino, diethylamino, 2-carboxyethylamino), an acylamino group having 1 to 10 (such as unsubstituted acetamide, benzamide), a sulfonylamino group having 1 to 8 carbon atoms (such as methylsulfonylamino, phenylsulfonylamino, butylsulfonylamino, n-octylsulfonylamino), a ureido group having 1 to 10 carbon atoms (such as unsubstituted ureido, methylureido), a urethane group having 2 to 10 carbon atoms (such as methoxycarbonylamino, ethoxycarbonylamino), an alkylthio group having 1 to 12 carbon atoms (such as methylthio, ethylthio, octylthio), an arylthio group having 6 to 12 carbon atoms (such as phenylthio, naphthylthio), an alkylsulfonyl group having 1 to 8 carbon atoms (such as methylsulfonyl, butylsulfonyl), an arylsulfonyl group having 7 to 12 carbon atoms (such as phenylsulfonyl, 2-naphtylsulfonyl), a sulfamoyl group having 0 to 8 carbon atoms (such as unsubstituted sulfamoyl, methylsulfamoyl), and a heterocyclic group (such as 4-pylidyl, piperidino, 2-furyl, furfuryl, 2-thienyl, 2-pyrrolyl, 2-quinolylmorpholine). Preferred examples of the substituent on R¹⁰ and R¹¹ in formula (VI) include an aryl group, an acyl group, an alkoxycarbonyl group, an aryloxycarbonyl group, a carbamoyl group, an alkoxy group, an aryloxy group, an acyloxy group, an acylamino group, a ureido group and a heterocyclic group.

The fluorine-containing polymer that can be preferably used in the present invention include at least one kind of fluoro-aliphatic group-containing monomer, and at least one kind of amide group-containing monomer, as the polymerization units. Two or more kinds of respective monomer may be contained as the polymerization units in the polymer. The polymer may be a copolymer containing one or more kinds of other copolymerizable monomer as a polymerization unit. For such an other copolymerizable monomer, those described in J. Brandrup, Polymer Handbook 2nd ed., Chapter 2, pp. 1-483, Wiley Interscience (1975), can be used. Examples thereof include compounds having at least one addition-polymerizable unsaturated bond selected from acrylates, methacrylates, (meth)acrylamides, allyl compounds, vinyl ethers and vinyl esters.

Specific examples thereof include the following monomers.

### Acrylates:

Methyl acrylate, ethyl acrylate, propyl acrylate, chloroethyl acrylate, 2-hydroxyethyl acrylate, trimethylol-propane monoacrylate, benzyl acrylate, methoxybenzyl acrylate, furfuryl acrylate and tetrahydrofurfuryl acrylate.

### Methacrylates:

Methyl methacrylate, ethyl methacrylate, propyl methacrylate, chloroethyl methacrylate, 2-hydroxyethyl methacrylate, trimethylolpropane monomethacrylate, benzyl methacrylate, methoxybenzyl methacrylate, furlfuryl methacrylate and tetrahydrofurfuryl methacrylate.

### Acrylamides:

Acrylamide, N-alkylacrylamide (the alkyl group is an alkyl group having from 1 to 3 carbon atoms, e.g., methyl, ethyl, propyl), N,N-dialkylacrylamide (the alkyl group is an alkyl group having from 1 to 3 carbon atoms), N-hydroxyethyl-N-methylacrylamide and N-2-acetamidoethyl-N-acetylacrylamide.

### Methacrylamides:

Methacrylamide, N-alkylmethacrylamide (the alkyl group is an alkyl group having from 1 to 3 carbon atoms, e.g., methyl, ethyl, propyl), N,N-dialkylmethacrylamide (the alkyl group is an alkyl group having from 1 to 3 carbon atoms), N-hydroxyethyl-N-methylmethacrylamide and N-2-acetamidoethyl-N-acetylmethacrylamide.

### Allyl Compounds:

Allyl esters (e.g., allyl acetate, allyl caproate, allyl caprylate, allyl laurate, allyl palmitate, allyl stearate, allyl benzoate, allyl acetoacetate, allyl lactate) and allyloxy ethanol.

### Vinyl Ethers:

Alkyl vinyl ether (e.g., hexyl vinyl ether, octyl vinyl ether, decyl vinyl ether, ethyl hexyl vinyl ether, methoxy ethyl vinyl ether, ethoxyethyl vinyl ether, chloroethyl vinyl ether, 1-methyl-2,2-dimethylpropyl vinyl ether, 2-ethylbutyl vinyl ether, hydroxyethyl vinyl ether, diethylene glycol vinyl ether, dimethylaminoethyl vinyl ether, diethylaminoethyl vinyl ether, butylaminoethyl vinyl ether, benzyl vinyl ether and tetrahydrofurfuryl vinyl ether.

### Vinyl Esters:

Vinyl butyrate, vinyl isobutyrate, vinyl trimethyl acetate, vinyl diethyl acetate, vinyl valerate, vinyl caproate, vinyl chloroacetate, vinyl dichloroacetate, vinyl methoxy acetate, vinyl butoxy acetate, vinyl lactate, vinyl-β-phenyl butyrate and vinyl cyclohexyl carboxylate.

### Dialkyl Itaconates:

Dimethyl itaconate, diethyl itaconate and dibutyl itaconate.
Dialkyl esters and monoalkyl esters of fumaric acid:

### Dibutyl Fumarate.

### Others:

Acrylonitrile, methacrylonitrile, maleylonitrile and styrene,

The amount of unit derived from the fluoroaliphatic group-containing monomer in the fluorine-based polymer is preferably from 25 to 99 mass %, based on total of polymer units in the fluorine-based polymer. More preferred rate is different depending on the structure of the fluoro-aliphatic group, and the polymerization unit of the monomer represented by formula (IV) is preferably from 50 to 99 mass % based on the whole polymerization units composing the fluorine polymer. The rate is more preferably from 60 to 97 mass %, and further preferably from 70 to 95 mass %. The amount of the polymerization unit of the monomer represented by formula (V) is preferably from 25 to 60 mass %, more preferably from 30 to 50 mass %, still more preferably from 35 to 45 mass %, based on the whole polymerization units comprising the fluorine-based polymer. Further, two or more kinds of units of the fluoro-aliphatic group-containing monomers may be included in one polymer, and both one or more kinds of units of the monomer represented by formula (IV) and one or more kinds of units of the monomer represented by formula (V) may be contained in the polymer. On the other hand, the polymerization unit of the monomer having an amide group is preferably from 3 to 70 mass % based on the whole polymerization units composing the polymer containing a fluorine atom; more preferably from 5 to 60 mass %.

The weight average molecular weight of the fluorine-based polymer that can be preferably used in the present invention is preferably from 2,000 to 100,000, more preferably from 3,000 to 80,000, further preferably from 4,000 to 60,000. The weight average molecular weight as used herein means a molecular weight determined by differential refractometer detection with a solvent THF in a GPC analyzer using a column, “TSKgel GMHxL”, “TSKgel G4000HxL” or “TSKgel G2000H×L” (trade names, manufactured by Tosoh Corp.), and expressed in terms of polystyrene.

The fluorine-based polymer that can be preferably used in the present invention can be produced by an ordinary method. For example, the polymer may be produced by polymerizing the above-described monomer such as a monomer having a fluoroaliphatic group, and a monomer having an amide group, in an organic solvent with the addition of a general-purpose radical polymerization initiator. Depending on the case, the polymer may be produced in the same manner as above by further adding another addition-polymerizable unsaturated compound. According to the polymerizability of each monomer, dropwise polymerization of performing the polymerization while adding dropwise the monomers and an initiator in a reactor is also effective for obtaining a polymer having a uniform composition. In addition, any method such as an anionic polymerization, a cationic polymerization, or an emulsification polymerization may be used depending on the kind of the monomers to be employed.

Specific examples of the structure of the fluorine-based polymer that can be preferably used in the present invention are set forth below, however, the present invention is not limited thereto. In formulae, the numerical value indicates the molar ratio of each monomer component and Mw indicates the weight average molecular weight.

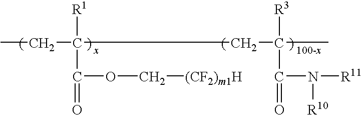

| | m¹ | R¹ | R³ | R¹¹ | R¹⁰ | x | Mw |
|---|---|---|---|---|---|---|---|
| P-1 | 4 | CH₃ | H | CH₃ | CH₃ | 60 | 1.9 × 10⁴ |
| P-2 | 4 | H | H | CH₃ | CH₃ | 80 | 1.4 × 10⁴ |
| P-3 | 6 | H | H | CH₃ | CH₃ | 70 | 2.8 × 10⁴ |
| P-4 | 6 | H | H | CH₃ | CH₃ | 80 | 1.6 × 10⁴ |
| P-5 | 6 | H | H | CH₃ | CH₃ | 90 | 1.8 × 10⁴ |
| P-6 | 8 | H | H | CH₃ | CH₃ | 75 | 8.2 × 10³ |
| P-7 | 8 | H | H | CH₃ | CH₃ | 95 | 4.6 × 10⁴ |
| P-8 | 6 | H | H | C₂H₅ | C₂H₅ | 85 | 1.5 × 10⁴ |
| P-9 | 6 | CH₃ | CH₃ | C₄H₉ (n) | C₄H₉ (n) | 80 | 1.9 × 10⁴ |
| P-10 | 6 | H | H | CH₂CH₂OCH₃ | CH₂CH₂OCH₃ | 90 | 1.2 × 10⁴ |

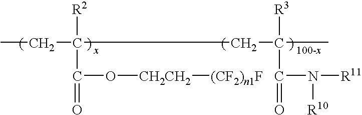

| | n¹ | R² | R³ | R¹¹ | R¹⁰ | x | Mw |
|---|---|---|---|---|---|---|---|
| P-11 | 4 | CH₃ | H | CH₃ | CH₃ | 55 | 8.8 × 10³ |
| P-12 | 4 | H | H | CH₃ | CH₃ | 40 | 1.3 × 10⁴ |
| P-13 | 6 | H | H | CH₃ | CH₃ | 40 | 1.7 × 10⁴ |
| P-14 | 6 | H | H | CH₃ | CH₃ | 35 | 2.1 × 10⁴ |
| P-15 | 6 | H | H | CH₃ | CH₃ | 45 | 9.0 × 10³ |
| P-16 | 8 | H | H | CH₃ | CH₃ | 30 | 1.5 × 10⁴ |
| P-17 | 6 | H | H | C₂H₅ | C₂H₅ | 40 | 1.5 × 10⁴ |
| P-18 | 6 | CH₃ | CH₃ | C₄H₉ (n) | C₄H₉ (n) | 40 | 1.9 × 10⁴ |
| P-19 | 6 | H | H | CH₂CH₂OCH₃ | CH₂CH₂OCH₃ | 40 | 1.2 × 10⁴ |
| P-20 | 6 | H | H | CH₂CH₂OH | CH₂CH₂OH | 40 | 1.1 × 10⁴ |

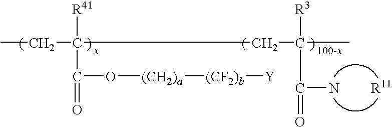

| | a | b | y | R⁴¹ | R³ | R¹¹ | x | Mw |
|---|---|---|---|---|---|---|---|---|
| P-21 | 1 | 4 | H | H | CH₃ | (CH₂)₄ | 80 | 1.5 × 10⁴ |
| P-22 | 1 | 6 | H | H | H | (CH₂)₅ | 85 | 1.3 × 10⁴ |
| P-23 | 1 | 6 | H | H | H | (CH₂)₂O(CH₂)₂ | 80 | 1.8 × 10⁴ |
| P-24 | 2 | 4 | F | H | CH₃ | (CH₂)₄ | 45 | 1.2 × 10⁴ |
| P-25 | 2 | 6 | F | H | H | (CH₂)₅ | 35 | 1.5 × 10⁴ |
| P-26 | 2 | 6 | F | H | H | (CH₂)₂O(CH₂)₂ | 40 | 2.3 × 10⁴ |
| P-27 | 3 | 6 | F | H | H | (CH₂)₆ | 40 | 1.7 × 10⁴ |
| P-28 | 6 | 6 | F | CH₃ | CH₃ | (CH₂)₂O(CH₂)₂ | 40 | 1.9 × 10⁴ |
| P-29 | 1 | 4 | H | H | CH₃ | C₆H₁₃ (n) | 90 | 2.0 × 10⁴ |
| P-30 | 1 | 6 | H | H | H | CH(CH₃)₂ | 85 | 1.3 × 10⁴ |
| P-31 | 1 | 6 | H | H | H | CH2CH2Ph | 80 | 1.8 × 10⁴ |
| P-32 | 2 | 4 | F | H | CH₃ | C₄H₉ (n) | 45 | 2.7 × 10⁴ |
| P-33 | 2 | 6 | F | H | H | CH(CH₃)₂ | 40 | 1.8 × 10⁴ |
| P-34 | 2 | 6 | F | H | H | C(CH₃)₂CH₂COCH₃ | 35 | 1.8 × 10⁴ |
| P-35 | 3 | 6 | F | H | H | CH₂OC₄H₉ (n) | 40 | 1.7 × 10⁴ |
| P-36 | 6 | 6 | F | CH₃ | CH₃ | C₄H₉ (t) | 45 | 1.9 × 10⁴ |

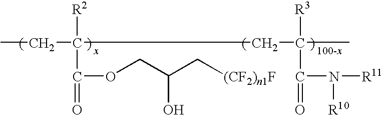

| | n¹ | R² | R³ | R¹¹ | R¹⁰ | x | Mw |
|---|---|---|---|---|---|---|---|
| P-37 | 4 | H | H | CH₃ | CH₃ | 45 | 1.1 × 10⁴ |
| P-38 | 4 | H | H | H | CH(CH₃)₂ | 40 | 1.3 × 10⁴ |
| P-39 | 6 | H | H | CH₃ | CH₃ | 40 | 1.8 × 10⁴ |
| P-40 | 6 | CH₃ | H | CH₃ | CH₃ | 35 | 2.7 × 10⁴ |
| P-41 | 6 | H | H | H | CH(CH₃)₂ | 40 | 1.4 × 10⁴ |
| P-42 | 6 | H | H | H | C(CH₃)₂CH₃COCH₃ | 40 | 1.9 × 10⁴ |

| | | | | | | |
|---|---|---|---|---|---|---|
| P-43 | 6 | H | H | 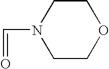 | 40 | 1.7 × 10⁴ |
| P-44 | 6 | CH₃ | CH₃ | 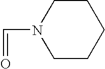 | 45 | 1.8 × 10⁴ |

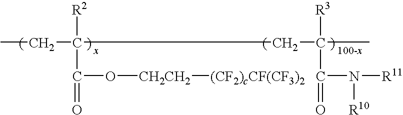

| | c | R² | R³ | R¹¹ | R¹⁰ | x | Mw |
|---|---|---|---|---|---|---|---|
| P-45 | 4 | H | H | CH₃ | CH₃ | 45 | 3.3 × 10⁴ |
| P-46 | 4 | H | H | H | CH(CH₃)₂ | 40 | 1.5 × 10⁴ |
| P-47 | 6 | H | H | CH₃ | CH₃ | 40 | 1.6 × 10⁴ |
| P-48 | 6 | CH₃ | H | H | CH(CH₃)₂ | 40 | 2.4 × 10⁴ |

| | | | | | | |
|---|---|---|---|---|---|---|
| P-49 | 6 | H | H | 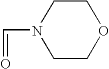 | 40 | 1.7 × 10⁴ |

| | Structure | Mw |
|---|---|---|
| P- 60 | 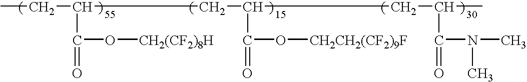 | 1.7 × 10⁴ |
| P- 51 | 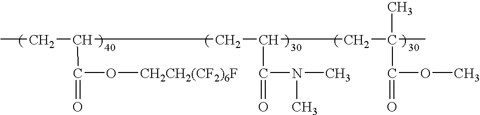 | 2.0 × 10⁴ |
| P- 52 | 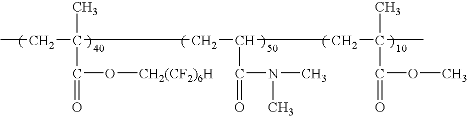 | 2.5 × 10⁴ |

In the dichroic dye composition of the present invention, only a single compound containing a fluorine atom may be used, or, alternatively, two or more compounds containing a fluorine atom may be used. The addition amount of the fluorine based compound is preferably within the range of 0.01 to 2 mass % with respect to addition amount of the dichroic dye; more preferably within the range of 0.05 to 1 mass %, and furthermore preferably within the range of 0.1 to 1 mass %.

Generally, a tilt angle of the dichroic dye at the air interface side can be adjusted by using the above-described horizontally orienting agent or another compound (for example, horizontally orientating agents disclosed in JP-A-2005-99248, JP-A-2005-134884, JP-A-2006-126768 and JP-A-2006-267183) to be added optionally, and the preferable horizontal orientated state can be realized as a polarizing element of the liquid crystal display device to which the light absorption anisotropic film of the present invention is applied.

In addition, the tilt angle of the dichroic dye at the alignment film side can be controlled by the above mentioned manner (for example, by using the agent for controlling a tilt angle at an alignment film, and the like).

### [Tilt Angle]

In the present invention, the term of “tilt angle” means an angle formed between a long axis of a dichroic dye molecule and an interface (alignment film interface or an air interface). Narrowing the tilt angle at the alignment film side to an extent and horizontally orientating provide preferable optical performance as the polarizing element efficiently. Accordingly, from the viewpoints of polarization performance, the tilt angle at the alignment film side is preferably from 0° to 100, further preferably from 0° to 5°, particularly preferably from 0° to 2°, and the most preferably from 0° to 1°. In addition, preferable tilt angle at the air interface side is from 0 degree to 10°, further preferably from 0 to 5°, particularly preferably from 0 to 2°. When the fluoro-aliphatic group-containing compound represented by formula (III) and/or the polymer containing at least one kind of polymerization unit of the fluoro-aliphatic group-containing monomer represented by formula (IV) or (V) and at least one kind of polymerization unit of the amide-group-containing monomer represented by formula (VI) is added to the dichroic dye composition of the present invention, it is possible to set the tilt angle of the dichroic dye at the alignment film side to smaller value, for example, 2° or less.

### [Alignment Film]

There have been provided the alignment film formed of various materials by various methods such as subjecting a film made of an organic compound (preferably a polymer) to a rubbing treatment, obliquely depositing an inorganic compound, forming a layer having microgrooves, or accumulating an organic compound (e.g., ω-trichosanic acid, dioctadecylmethylammonium chloride, methyl stearate) by Langmuir-Blodgett method (LB film). Alignment films having an alignment effect under an electric or magnetic field or irradiation are also known. According to the present invention, any alignment films, which can contribute aligning the dichroic dye of the light absorption anisotropic film provided on the alignment film, may be used, and, however, among them, alignment films prepared by subjecting a film of a polymer to a rubbing treatment are preferred from the view of controllability of a tilt angle at an alignment film interface. The rubbing treatment is usually performed by rubbing the surface of the polymer layer in a direction several times with a paper or a cloth. And it is especially preferred that the rubbing treatment is carried out according to the method described in “Handbook of liquid Crystal (Ekisyo Binran)” published by MARUIZEN CO., Ltd.

The thickness of the alignment film is preferably from 0.01 to 10 μm, and more preferably from 0.05 to 1 μm.

Various types of polymers which can be used for producing alignment films are described in various documents, and various polymers are commercially available. According to the present invention, alignment layers formed of polyvinyl alcohols or derivatives thereof are preferably used. Especially, alignment films formed of modified polyvinyl alcohols bonding with hydrophobic groups are preferable. Regarding various matters of the alignment film, it is possible to refer to the descriptions from line 24 of p. 43 to line 8 of p. 49 in WO01/88574A1.

### [Rubbing-Density of Alignment Film]

It is possible to vary a rubbing-density of an alignment film by a method described in “Handbook of liquid Crystal (Ekisyo Binran)” published by MARUIZEN CO., Ltd. A rubbing-density (L) is quantified by a formula (A) below.

L=N1{1+(2πrn/60v)} Formula (A)

In formula (A), N is a number of rubbing, 1 is a contact length of a rubbing-roller, r is a roller-radius, n is revolutions per minute (rpm) and v is moving velocity (per second).

The rubbing-density may be increased by increasing the number of rubbing, lengthening the contact length of the rubbing roller, increasing radius of the roller, increasing revolutions per minute of the roller and/or decreasing moving velocity. On the other hand, the rubbing-density may be decreased by doing the reverse thereof.

There is a relationship between a rubbing-density and a pre-tilt angle of the alignment film that the pre-tilt angle is decreased as the rubbing-density is higher, and the pre-tilt angle is increased as the rubbing-density is lower.

### [Support]

The support to be used for the present invention may be either a transparent support or an opaque support with an aide of coloring or so. The support is preferably transparent, and, in particular, preferably has a light transmission of 80% or more. The support is preferably selected from films formed of optically isotropic polymers. Examples of such polymers or preferred embodiments of the support are same as those described in paragraph No [0013] in JP-A-2002-22942. The films formed of the polymers, which are commonly known as easy to develop birefringence, such as polycarbonates or polysulfones, may be also used after being modified by the process described in WO00/26705 thereby to reduce the development of birefringence.

Polymer films of cellulose acetates having an acetylation rate from 55.0% to 62.5%, preferably from 57.0% to 62.0%, are preferably employed in the present invention. The preferred scope of acetylation rates and the preferred chemical structures of cellulose acetates are same as those described in paragraph No. [0021] in JP-A-2002-196146. It is disclosed in Journal of Technical Disclosure (Hatsumei Kyoukai Koukai Gihou) No. 2001-1745, published by Japan Institute of Invention and Innovation, cellulose acylate films produced by using chlorine-free solvents, and the cellulose acetate films described therein can be employed in the present invention.

The preferred scopes of the depth-retardation value and the birefringence value of the cellulose acetate film to be used as a transparent support are described in paragraph Nos. [0018] to [0019] in JP-A-2002-139621.

In order to control the retardation of a polymer film as the transparent support, especially a cellulose acetate film, aromatic compounds having at least two aromatic rings may be used as an agent for increasing retardation. The preferred scope and the preferred amount of the aromatic compound are same as those describe in paragraph Nos. [0021] to [0023] in JP-A-2002-139621. Examples of such an agent for increasing retardation are described in WO01/88574, WO00/2619, JP-A-2000-111914, JP-A-2000-275434, JP-A-2002-363343 or the like.

The cellulose acylate film, produced by a solvent-casting method using a cellulose acylate solution (dope), is preferably used. The dope may further comprise the agent for increasing retardation, and such a dope is preferred. Multilayered films can be produced by using the cellulose acylate solution (dope). The production of the films can be carried out according to the descriptions in paragraph Nos. [0038] to in JP-A-2002-139621.

Stretching treatment of the cellulose acetate film may be carried out in order to control its retardations. The stretch ratio is desirably from 3% to 100%. The cellulose acetate film is preferably stretched by tenders. For controlling the slow axis of the film to high accuracy, the deference in velocities, departure times and the like between of the left and right tenter clips are preferably as small as possible.

Plasticizes may be added to the cellulose acetate films in order to improve the mechanical properties of the films and the drying speed. Examples of the plasticizer and the preferred scope of the plasticizers are same as those described in paragraph Nos. [0043] in JP-A-2002-139621.

Anti-degradation agents such as antioxidants, decomposers of peroxides, inhibitors of radicals, in-activators of metals, trapping agents of acids or amines, and UV ray protective agents, may be added to the cellulose acetate film. The anti-degradation agents are described in paragraph No. [0044] in JP-A-2002-139621. The preferred example of the anti-degradation agent is butylated hydroxy toluene (BHT). The UV ray protective agents are described in JP-A-7-11056.

Surface treatment or measurement of solid-surface energy for the cellulose acylate film can be carried out according to the descriptions in paragraph Nos. [0051] to in JP-A-2002-196146.

The preferred thickness of the cellulose acylate film may vary depending on the application of the film, and, in usually, the thickness of the film is preferably from 5 to 500 μm, more preferably from 20 to 250 μm and most preferably from 30 to 180 μm. Especially, for being used in optical applications, the thickness of the cellulose acylate film is preferably from 30 to 110 μm.

### [Usage of Light Absorption Anisotropic Film]

The light absorption anisotropic film of the present invention will function as a polarizing film whereby a linearly polarized light, circularly polarized light or oval polarized light can be obtained by utilizing the anisotropy in light absorption and further is capable of providing functions as various anisotropic films such as refractive anisotropy and conductivity anisotropy by selecting the film-forming process, the support and the composition containing the dye, whereby it can be made various types of polarizing elements which can be used for various purposes.

The polarizing element of the present invention can be produced by: (1) a step of rubbing a support or an alignment film formed on a support; (2) a step of applying the dichroic dye composition of the present invention dissolved in an organic solvent on the rubbing treated support or alignment film; and (3) a step of orientating the dicliroic dye composition by causing the organic solvent to evaporate. Detailed information about the steps (1) to (3) are as the foregoing description.

In a case where the light absorption anisotropic film of the present invention is formed on a support to use as a polarizing element, the formed light absorption anisotropic film itself may be used, or not only the above-mentioned protective layer but also layers having various functions such as an adhesive layer and a reflection-preventing layer, an oriented film, and layers having optical functions such as a function as a phase difference film, a function as a brightness-improved film, a function as a reflective film, a function as a semi-transmissive reflective film and a function as a diffusion film may be formed by lamination by e.g. a wet film-forming method, so that it may be used in the form of a laminate.

Such layers having optical functions may be formed, for example, by the following methods.

A layer having a function as a phase difference film may be formed by applying a stretching treatment as disclosed in e.g. Japanese Patent No. 2841377 or Japanese Patent No. 3094113, or by applying a treatment as disclosed in e.g. Japanese Patent No. 3168850.

Further, a layer having a function as a brightness-improved film may be formed by forming ultrafine pores by a method as disclosed in e.g. JP-A-2002-169025 or JP-A-2003-29030, or by superposing two or more cholesteric liquid crystal layers with different central wavelengths of the selective reflection.

A layer having a function as a reflective film or a semi-transmissive reflective film may be formed by using a metal thin film obtained by deposition or sputtering.

A layer having a function as a diffusion film may be formed by coating the above protective layer with a resin solution containing fine particles.

Further, a layer having a function as a phase difference film or an optical compensation film may be formed by applying a liquid crystalline compound such as a discotic liquid crystalline compound and orienting it.

A formation of the dichroic dye composition of the present invention by coating enables to produce In-Cell type polarizer. In this occasion, In-Cell type polarizer having high voltage retention rate (electric charge retention property) suitable for active drive can be obtained by suppressing the value of an electric conductivity and/or a sodium ion concentration of the dichroic dye composition not larger than a constant value. As a result, a liquid crystal element using this In-Cell type polarizer superior in driving performance and display performance can be obtained.

In the present invention, the electrical conductivity of the dichroic dye composition is preferably 25 mS/cm or less, more preferably 10 mS/cm or less, and further preferably is 1 mS/cm or less. Since the electrical conductivity is within such a range, an in-cell polarizer having a high voltage retention ratio can be produced. The electrical conductivity is generally 0.2 mS/cm or more. A too high electrical conductivity leads to an undesirable increase in solubility of polar impurities.

The electrical conductivity of the composition for the in-cell polarizer is measured by a two-electrode method or four-electrode method using a conductivity meter. Although the electrical conductivity can be measured by several methods defined in “Method of Testing Industrial Water”, JIS K0101:1998, the four-electrode method is preferred to the two-electrode method in the view point of high measurement accuracy by guard electrodes. In order to avoid adverse effects of careers (e.g. ions) localized by an applied voltage (potential difference generated between the electrodes) in the vicinity of electrodes, measurement by applying alternate current (AC) is preferred to that by direct current (DC).

As described above, the dichroic dye composition of the present invention is a mixture (solution) of a dye, a solvent, and some additives such as a surfactant used as required. Chemical species of components in the mixture and impurities derived from these components, in particular polar components and ionic components contribute to electrical conductivity of the composition. Reducing these contents ensures a lower electrical conductivity.

Regarding with a technique about reducing electric conductivity of the dichroic dye composition of the present invention, it is disclosed in, for example, JP-A-2006-309185, which is applicable to the present invention.

In the present invention, the sodium ion concentration of the dichroic dye composition of the present invention is preferably 2500 ppm or less, more preferably 1000 ppm or less, and further preferably 100 ppm or less. The lower limit is usually 10 ppm or more. Too high sodium ion concentration leads to undesirable elution into the liquid crystal element (liquid crystal layer) and an adverse affect to electrical characteristics. For example, the sodium ion concentration is controlled within the above-described range by the several methods that are shown to reduce impurities as described above.

The sodium ion concentration in the dichroic dye composition of the present invention is determined by a combination of an ion-selective electrode of which electrical potential varies in response to the concentration of specific ions in the composition and a reference electrode. Although, it can be measured by several methods, such as flame photometry, flame atomic absorption spectrometry, and ion chromatography that are defined in “Method of Testing Industrial Water” in JIS K0101:1998, the ion-electrode measuring method defined in “General Rule of Method of Measuring by Ion Electrode”, JIS K0122:1998 is preferred because a comparatively high sodium ion concentration can be measured directly. Use of an ion meter with an ion-selective electrode without pH adjusting by a buffer solution is more preferred because a change in dissociation state by pH can be suppressed.

The light absorption anisotropic film may be formed directly, for example, onto the surface of the protection film for a polarizing plate by a coating method. Also, it may be formed by transferring transfer materials which will be explained below. Namely, one example of the method for producing the liquid crystal display device of the present invention include a formation of a light absorption anisotropic layer comprising the light absorption anisotropic film by transferring from the transfer materials. The formation of the light absorption anisotropic layer using the transfer materials enables to fabricate the liquid crystal display device having favorite display performance with simple manner reducing the number of processes thereof.

An explanation about the transfer materials which are useful for forming the light absorption anisotropic layer in the liquid crystal display device will be described below.

### [Transfer Material]

The transfer material which can be used in the present invention is composed of, for example, at least a support and a optically anisotropic layer as shown in FIG. 3(a) of JP-A-2007-279705. It is preferable that the transfer material is further composed of at least a photosensitive resin layer, as shown in FIG. 3(b) of JP-A-2007-279705. The photosensitive resin layer is useful because it makes the transfer of the optically anisotropic layer easy even though it does not pass through a process of patterning or so. Further, the transfer material may be composed of a layer for controlling dynamics performance such as cushioning property in order to absorb the roughness on the substrate to be transferred or for adding a roughness subordination in the occasion of transferring between the support and the optically anisotropic layer; as shown in, for example, FIG. 3(c) of JP-A-2007-279705. Furthermore, a layer functioning as an alignment layer for controlling the orientation of the dichroic dye in the optically anisotropic layer may be disposed as shown in FIG. 3(d) of JP-A-2007-279705; and the transfer material may be composed of both of the above layers as shown in FIG. 3(e) of JP-A-2007-279705. Also, as shown in FIG. 3(f) of JP-A-2007-279705, a protective layer capable of being pealed apart may be provided onto the uppermost surface with the purposes of surface protection of the photosensitive resin layer or so.

### [Support]

The support used as the above-described transfer material is not particularly limited and may be transparent or opaque. Examples of the polymer, which can constitute the support, include cellulose esters (for example, cellulose acetate, cellulose propionate, cellulose butyrate, cellulose acetate propionate, cellulose acetate butyrate), polyolefins (for example, norbornene based polymer), poly(meth)acrylates (for example, polymethylmethacrylate), polycarbonates, polyesters, and polysulfones. For the purpose of property examination in a manufacturing process, the support is preferably composed of a transparent and low-birefringence material. From the viewpoint of the low-birefringence property, cellulose ester films and norbornene based polymer films are preferable. Commercially available norbornene based polymers, ARTON (trade name, manufactured by JSR), ZEONEX, ZEONOR (trade names, manufactured by ZEON CORPORATION) may be used. Polycarbonate, poly(ethylene terephthalate), or the like which is inexpensive, may also be preferably used.

### [Optically Anisotropic Layer]

The optically anisotropic layer in the transfer material does not need to satisfy a sufficient optical performance for polarization capability. It may be, for example, a layer in which the polarization capability reveals or changes through an exposure process executed in the transferring process, and finally exhibits the polarization capability which is necessary for the polarizing film.

### [Photosensitive Resin Layer]

The transfer material may be formed of a photosensitive resin layer. The photosensitive resin layer is preferably formed of a photosensitive resin composition comprising at least (1) an alkali-soluble resin, (2) a monomer or oligomer, and (3) a photopolymerization initiator or photopolymerization initiator series.

### (1) Alkali-Soluble Resin

As the alkali-soluble resin (herein, also simply referred to as “binder”), preferred are polymers having a polar group such as a carboxylic acid group or a carboxylate group at its side chain. Examples of the polymer include a methacrylic acid copolymer, an acrylic acid copolymer, an itaconic acid copolymer, a crotonic acid copolymer, a maleic acid copolymer, and a partially esterified maleic acid copolymer described in, for example, JP-A-59-44615, JP-B-54-34327 (“JP-B” means examined Japanese patent publication), JP-B-58-12577, JP-B-54-25957, JP-A-59-53836, and JP-A-59-71048. The examples further include a cellulose derivative having a carboxylic acid group at its side chain. In addition to the foregoing, a product obtained by adding a cyclic acid anhydride to a polymer having a hydroxyl group can also be preferably used. In addition, particularly preferable examples include a copolymer of benzyl (meth)acrylate and (meth)acrylic acid and a multi-component copolymer of benzyl (meth)acrylate, (meth)acrylic acid, and any other monomer described in U.S. Pat. No. 4,139,391. These binder polymers having a polar group may be used singly or in the form of a composition containing the binder polymer together with an ordinary film-forming polymer. The addition amount of the binder polymer is generally in the range of from 20 to 50 mass %, preferably from 25 to 45 mass %, based on the whole solid content of the photosensitive resin composition.

### (2) Monomer or Oligomer

The monomer or the oligomer contained in the photosensitive resin composition is preferably a monomer or oligomer which has two or more ethylenically unsaturated double bonds and which undergoes addition-polymerization by irradiation with light. The monomer or oligomer may be a compound having at least one addition-polymerizable ethylenically unsaturated group therein and having a boiling point of 100° C. or higher at normal pressure. Examples thereof include: a monofunctional acrylate and a monofunctional methacrylate such as polyethylene glycol mono(meth)acrylate, polypropylene glycol mono(meth)acrylate, and phenoxyethyl(meth)acrylate; polyethylene glycol di(meth)acrylate, polypropylene glycol di(meth)acrylate, trimethylolethane triacrylate, trimethylolpropane tri(meth)acrylate, trimethylolpropane diacrylate, neopentyl glycol di(meth)acrylate, pentaerythritol tetra(meth)acrylate, pentaerythritol tri(meth)acrylate, dipentaerythritol hexa(meth)acrylate, dipentaerythritol penta(meth)acrylate, hexanediol di(meth)acrylate, trimethylolpropane tri(acryloyloxypropyl)ether, tri(acryloyloxyethyl)isocyanurate, tri(acryloyloxyethyl)cyanurate, glycerin tri(meth)acrylate; a polyfunctional acrylate or polyfunctional methacrylate which may be obtained by adding ethylene oxide or propylene oxide to a polyfunctional alcohol such as trimethylolpropane or glycerin and converting the adduct into a (meth)acrylate.

Examples of the monomer and the oligomer further include urethane acrylates as described in JP-B-48-41708, JP-B-50-6034, and JP-A-51-37193; and polyester acrylates as described in JP-A 48-64183, JP-B-49-43191, and JP-B-52-30490; polyfunctional acrylates or polyfunctional methacrylates such as an epoxy acrylate which is a reaction product of an epoxy resin and (meth)acrylic acid.

Among these, trimethylolpropane tri(meth)acrylate, pentaerythritol tetra(meth)acrylate, dipentaerythritol hexa(meth)acrylate, and dipentaerythritol penta(meth)acrylate are preferable.

Further, other than the above, “polymerizable compound B” described in JP-A-11-133600 can be mentioned as a preferable example.

These monomers or oligomers may be used singly or as a mixture of two or more kinds thereof. The content of the monomer or the oligomer is generally in a range of from 5 mass % to 50 mass %, preferably from 10 mass % to 40 mass %, based on the total solid content of the photosensitive resin composition.

### (3) Photopolymerization Initiator or Photopolymerization Initiator Series

Examples of the photopolymerization initiator or the photopolymerization initiator series (in the present specification, the term “photo-polymerization initiator series” means a polymerization initiating mixture that exhibits a function of photo-polymerization initiation with a plurality of compounds combined with each other) include vicinal polyketaldonyl compounds disclosed in U.S. Pat. No. 2,367,660, acyloin ether compounds described in U.S. Pat. No. 2,448,828, aromatic acyloin compounds substituted by an α-hydrocarbon described in U.S. Pat. No. 2,722,512, polynuclear quinone compounds described in U.S. Pat. No. 3,046,127 and U.S. Pat. No. 2,951,758, combinations of triarylimidazole dimer and p-aminoketone described in U.S. Pat. No. 3,549,367, benzothiazole compounds and trihalomethyl-s-triazine compounds described in JP-B-51-48516, trihalomethyl-triazine compounds described in U.S. Pat. No. 4,239,850, and trihalomethyloxadiazole compounds described in U.S. Pat. No. 4,212,976. In particular, trihalomethyl-s-triazine, trihalomethyloxadiazole, and triarylimidazole dimer are preferable.

In addition, “polymerization initiator C” described in JP-A-11-133600 can also be mentioned as preferable examples.

These photopolymerization initiators and photopolymerization initiator series each may be used singly. Alternatively, a mixture of two or more selected from these photopolymerizable initiators and photopolymerization initiator series may be used. In particular, it is preferable to use two or more selected from photopolymerizable initiators and photopolymerization initiator series. When two or more selected from photopolymerizable initiators and photopolymerization initiator series are used, the display property, particularly evenness of display, can be improved.

As to the content of the photo-polymerization initiator and the photo-polymerization initiator series, the content is generally in the range of from 0.5 to 20 mass %, preferably from 1 to 15 mass %, based on the total solid content of the photosensitive resin composition.

### [Other Layers]

Between the support and the optically anisotropic layer of the transfer material, a thermoplastic resin layer to control mechanical characteristics and conformity to irregularity may be preferably provided. The component used in the thermoplastic resin layer is preferably an organic polymer substance described in JP-A-5-72724. The substance can be preferably selected from organic polymer substances having a softening point of about 80° C. or lower according to the Vicat method (specifically, the method of measuring a polymer softening point according to American Material Test Method ASTMD 1235). Specifically, the substance may be an organic polymer, and examples thereof include: a polyolefin such as polyethylene or polypropylene; an ethylene copolymer such as a copolymer of ethylene and vinyl acetate or a saponified product thereof; a copolymer of ethylene and acrylic acid ester or a saponified product thereof; polyvinyl chloride; a vinyl chloride copolymer such as a copolymer of vinyl chloride and vinyl acetate or a saponified product thereof; polyvinylidene chloride; a vinylidene chloride copolymer; polystyrene; a styrene copolymer such as a copolymer of styrene and (meth)acrylic acid ester or a saponified product thereof; polyvinyl toluene; a vinyltoluene copolymer such as a copolymer of vinyltoluene and (meth)acrylic acid ester or a saponified product thereof; poly(meth)acrylic acid ester; a (meth)acrylic acid ester copolymer such as a copolymer of butyl (meth)acrylate and vinyl acetate; and a polyamide resin such as a vinyl acetate copolymer nylon, a copolymerized nylon, N-alkoxymethylated nylon, and N-dimethylaminated nylon.

In the transfer material of in the present invention, it is preferable to provide an intermediate layer so as to prevent mixing of components during application of a plurality of coating layers and during storage after the application. The intermediate layer is preferably an oxygen blocking film having oxygen blocking function described as “a separating layer” in JP-A-5-72724. By using such an oxygen blocking film, the exposure sensitivity is heightened, the time load of the exposing machine is decreased, and the productivity is improved. The oxygen blocking film is preferably a film with a low oxygen permeability and is dispersible or soluble in water or an aqueous alkaline solution. Such a film may be properly selected from ordinary oxygen blocking films. Among them, a combination of polyvinyl alcohol and polyvinylpyrrolidone is particularly preferable.

The above thermoplastic resin layer or the above intermediate layer can be also used as the above alignment layer. In particular, polyvinyl alcohol and polyvinylpyrrolidone which are preferably used for the intermediate layer is also effective as the alignment layer and it is preferable to make the functions of the intermediate layer and the alignment layer into one layer.

It is preferable to provide a thin protective film on the resin layer in order to protect the resin layer from pollution or damage at storage. The protective film may comprise a material which is the same as or similar to that of the temporary support, but the protective film should be easily separated from the resin layer. The protective film material may be, for example, silicon paper, polyolefin sheet or polytetrafluoroethylene sheet.

The individual layers of the optically anisotropic layer, photosensitive resin layer, and optionally-formed alignment layer, thermoplastic resin layer, and intermediate layer can be formed by coating such as dip coating, air knife coating, curtain coating, roller coating, wire bar coating, gravure coating and extrusion coating (U.S. Pat. No. 2,681,294). Two or more layers may be coated simultaneously. As to the simultaneous coating methods, descriptions thereof can be found in U.S. Pat. Nos. 2,761,791, 2,941,898, 3,508,947 and 3,526,528, and Yuji Ijarasaki, Coating Kogaku (Coating Engineering), p. 253, Asalkura Shoten Co., Ltd. (1973).

[Formation Method of Optically Anisotropic Layer with Use of Transfer Material]

Methods of transferring the transfer material on a substrate are not specifically limited, so far as the optically anisotropic layer and the photosensitive resin layer can be simultaneously transferred onto the substrate. For example, the transfer material in a film form may be attached to the substrate so that the surface at the photosensitive resin layer of the transfer material is faced to the surface of the substrate, then pressing under heating or no-heating with rollers or flat plates of a laminator. Specifically, laminators and laminating methods described in the following documents may be used: JP-A-7-110575, JP-A-11-77942, JP-A-2000-334836, and JP-A-2002-148794. From the viewpoint of suppression of contamination by foreign substances, it is preferable to use the method described in JP-A-7-110575. Afterwards, the support may be pealed apart, and another layer, for example, an electrode layer may be formed on the surface of the optically anisotropic layer which is exposed by being pealed apart.

The substrate being a target transfer material onto which the transfer material is transferred is not particularly specified. For example, a transparent substrate is used as the target transfer material Examples thereof include known glass plates such as a soda glass plate having a silicon oxide film on its surface, a low-expansion glass, a non-alkali glass, and a quartz glass plate, and a plastic film. The target transfer material may be also a material made by providing a layer such as a solid optically anisotropic layer al over a transparent support. By subjecting the target transfer material to a coupling treatment in advance, adhesion of the target transfer material to the photosensitive resin layer can be improved. The method described in JP-A-2000-39033 is preferable as the coupling treatment. The thickness of the substrate is not particularly limited, and is preferably 700 to 1200 μm in general.

It may be not providing an adhesive layer onto the optically anisotropic layer, but providing the adhesive layer onto the target transfer material.

The present invention can provide a dye composition having both the liquid crystallinity and high dichroism. Also, the present invention can provide a light absorption anisotropic film, a polarizing element and a liquid crystal display device all employing the dye composition.

The present invention will be described in more detail based on the following examples, but the present invention is not limited thereto.

### EXAMPLES

In the following examples, the measurements regarding with the optical performance of the light absorption anisotropic film were executed as described below.

### <Dichroic Ratio>

A dichroic ratio was calculated using the following equation after measuring an absorbance of the light absorption anisotropic film with a spectral photometer arranging an iodine series polarizing element in an incident light optical system.

Dicliroic Ratio (D)=Az/Ay

Az: Absorbance of a light absorption anisotropic film for a polarized light in the absorption axis direction
Ay: Absorbance of a light absorption anisotropic film for a polarized light in the polarization axis direction

### Example 1

Adding 2 mass parts of the azo dye No. (A-16) into 98 mass parts of chloroform, the resultant mixture was stirred and dissolved followed by filtering and as a result, a coating liquid of a dichroic dye composition was obtained. Subsequently, the above coating liquid was applied onto a rubbed alignment film formed on a glass substrate; and then, the coated film was naturally dried using chloroform at a room temperature. As the alignment film, polyimide (trade name: SE-150, manufactured by Nissan Chemical Industries, Ltd.) was used.

The dichroic ratio (D) calculated from absorbance of the dye film for a polarized light having a vibration plane in the absorption axis direction (Az) and absorbance of the dye film for a polarized light having a vibration plane in the polarization axis direction (Az), maximum absorption wavelength (X max) and phase transition temperatures of the resultant light absorption anisotropic film are shown in Table 1. The composition had a nematic liquid crystallinity, and a high dichroic ratio (light absorption anisotropic property) sufficiently functional as the polarizing film.

### Example 2

The light absorption anisotropic films were prepared in the same manner as Example 1 except that the azo dye was replaced to the azo dye Nos. (A-38), (A-46), (A-52), (A-53) or (A-55), respectively.

The dichroic ratio (D), the maximum absorption wavelength (λ max) and the phase transition temperatures of each of the resultant light absorption anisotropic films are shown in Table 1. The compositions each had a nematic liquid crystallinity, and a high dichroic ratio sufficiently functional as the polarizing film.

### Example 3

Adding 2 mass parts of the azo dye No. (A-38) and 0.16 mass parts of Iragacure OXE-01 (trade name, manufactured by Ciba Specialty Chemicals Ltd.) as a polymerization initiator into 97.84 mass parts of chloroform, the resultant mixture was stirred and dissolved followed by filtering and as a result, a coating liquid of a dichroic dye composition was obtained. The above coating liquid was applied onto the alignment film in the same condition as Example 1, and after the coated film was naturally dried, the oriented state was fixed by irradiating ultra-violet ray of 2J. The dichroic ratio (D), the maximum absorption wavelength (λ max) and phase transition temperatures of the resultant light absorption anisotropic film are shown in Table 1. The composition had a nematic liquid crystallinity, and the resultant anisotropic dye film had a high dichroic ratio sufficiently functional as the polarizing film.

### Comparative Example 1

The light absorption anisotropic film was prepared in the same manner as Example 1 except that the azo dye was replaced to the azo dye No. (6) being described in JP-A-11-305036.

The dichroic ratio (D), the maximum absorption wavelength (λ max) and phase transition temperatures of the resultant light absorption anisotropic film are shown in Table 1. Although the composition had a nematic liquid crystallinity, crystallization occurred on the alignment film and any anisotropic property was not exhibited.

TABLE 1

| | | | Maximum absorption | Phase transition |
|---|---|---|---|---|
| | Axo dye No. | Dichroic Ratio (D) | wavelength (nm) | temperature (° C.) |
| Example 1 | A-16 | 48.9 | 540 | K 137° C. N 266° C. I |
| Example 2 | A-38 | 20.5 | 490 | K 143° C. N 216° C. I |
| Example 2 | A-46 | 28.1 | 530 | K 191° C. N 262° C. I |
| Example 2 | A-52 | 22.5 | 540 | K 162° C. N 250° C. I |
| Example 2 | A-53 | 4.0 | 561 | K 185° C. N 210° C. I |
| Example 2 | A-55 | 8.2 | 560 | K 173° C. N 186° C. I |
| Example 3 | A-38 | 20.5 | 490 | K 240° C. N 216° C. I |
| Comparative example 1 | Azo dye No. (6) desribed in | Not orientated because of | 490 | K 240° C. N 280° C. I |
| | JP-A-11-305036 | crystallization | | |

K: Crystalline Phase
N: Nematic Phase
I: Isotropic Phase

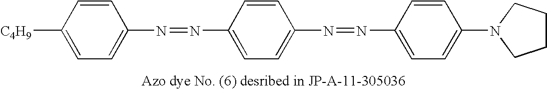

### Example 4

The light absorption anisotropic films were prepared in the same manner as Example 1 except that the azo dye was replaced to the azo dye Nos. (A-47) and (A-57), respectively. The dichroic ratio (D), the maximum absorption wavelength (λmax), the phase transition temperatures and the tilt angle (β) of each of the resultant light absorption anisotropic films are shown in Table 2. The term of “tilt angle (P)” means an angle formed between a long axis of a molecule and a substrate plane, and it should be measured by radiating the incident light with the wavelength of λ nm into a sample such as film or so by means of AxoScan (trade name, manufactured by Axometrics Inc.). Radiating the incident light with the wavelength of λ nm from the direction slanted with 10° step from the normal direction to 50° in both sides with respect to the normal direction of the film, and making a slow axis and a phase lead axis in the plane determined by AxoScan as a slanted axis (rotation axis), the retardation values are measured at totally 11 points. AxoScan calculates the β value on the basis of the measured retardation value, an assumption value of an averaged refractive index and an input film thickness value. In this specification, β is measured using a light having a wavelength of 644 nm unless otherwise specified.

In the above measurement, the assumption value of the averaged refractive index may be employable from Polymer Handbook (JOHNWILEY & SONS, INC), Liquid Crystal Database LiqCryst (LCI Publisher GmbH), and values in catalogs of various optical compensation films.

In addition, regarding with film whose average refractive index is unknown; the value can be measured by means of Abbe's refractometer.

### Example 5

Adding 2 mass parts of the azo dye No. (A-47) and 0.012 mass parts of the horizontally orientating agent No. (I-6) into 97.988 mass parts of chloroform, and the resultant mixture was stirred and dissolved, followed by filtration to obtain a dichroic dye composition coating liquid. The coating liquid was applied and dried in the same condition as Example 1. The dichroic ratio (D), the maximum absorption wavelength (λmax), the phase transition temperatures and the tilt angle (β) of the resultant light absorption anisotropic film are shown in Table 2.

From the results of Example 4 and Example 5, it is verified that the compositions each had a nematic liquid crystallinity, and has a high dichroic ratio sufficiently functionable as the polarizing film. Further, because the light absorption anisotropic film formed by the composition including the dichroic dye and the fluoro-aliphatic group-containing compound represented by formula (III) had the dichroic dye substantially orientating horizontally, a polarizing film with a large dichroic ratio can be realized.

### Example 6

The light absorption anisotropic film was prepared in the same manner as Example 5 except that the azo dye was replaced to the azo dye No. (A-57) and the horizontally orientating agent was replaced to the horizontally orienting agent No. (P-13). The dichroic ratio (D), the maximum absorption wavelength (λmax), the phase transition temperatures and the tilt angle (β) of the resultant light absorption anisotropic film are shown in Table 2.

From the results of Example 4 and Example 6, it is verified that the compositions each had a nematic liquid crystallinity, and has a high dichroic ratio sufficiently functionable as the polarizing film. Further, because the light absorption anisotropic film formed by the composition including the dichroic dye and the polymer containing the polymerization unit of the monomer represented by formula (V) and the polymerization unit of the monomer represented by formula (VI) had the dichroic dye substantially orientating horizontally, a polarizing film with a large dichroic ratio can be realized.

TABLE 2

| | | Horizontally | | | | |
|---|---|---|---|---|---|---|
| | Azo dye | orienting | Dichroic | Maximum absorption | Phase transition | Tilt angle |
| | No. | agent No. | Ratio (D) | wavelength (nm) | temperature (° C.) | (β) |
| Example 4 | A-47 | None | 45.1 | 530 | K 134° C. N 219° C. I | 9° |
| Example 4 | A-57 | None | 16.1 | 523 | K 141° C. N 258° C. I | 6° |
| Example 5 | A-47 | I-6 | 48.5 | 535 | K 134° C. N 219° C. I | 2° |
| Example 6 | A-57 | P-13 | 25.3 | 530 | K 141° C. N 258° C. I | 2° |

K: Crystalline Phase
N: Nematic Phase
I: Isotropic Phase

Having described our invention as related to the present embodiments, it is our intention that the present invention not be limited by any of the details of the description, unless otherwise specified, but rather be construed broadly within its spirit and scope as set out in the accompanying claims.

This non-provisional application claims priority under 35 U.S.C. § 119 (a) on Patent Application No. 2008-092412 filed in Japan on Mar. 31, 2008, which is entirely herein incorporated by reference.

1. A dichroic dye composition, comprising at least one kind of azo dye represented by formula (I) that has a liquid crystallinity: 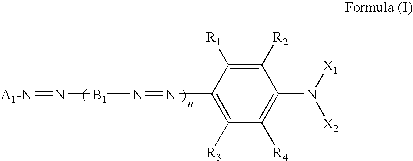
wherein R₁, R₂, R₃, R₄, X₁ and X₂ each independently represent a hydrogen atom or a substituent; A₁ is a substituted or unsubstituted phenyl group, a substituted or unsubstituted naphthyl group, or a substituted or unsubstituted aromatic heterocyclic group; B₁ is a divalent substituted or unsubstituted aromatic hydrocarbon group or a divalent substituted or unsubstituted aromatic heterocyclic group; n represents an integer of 1 to 5; and at least one of B₁s represents a phenylene group having an alkyl group.

2. The dichroic dye composition according to claim 1, wherein, in formula (I), A₁ represents a substituted or unsubstituted phenyl group; B₁ represents a divalent substituted or unsubstituted phenylene group; and n represents an integer of 2 to 4.

3. The dichroic dye composition according to claim 1, wherein the azo dye represented by formula (I) is a compound represented by formula (II): 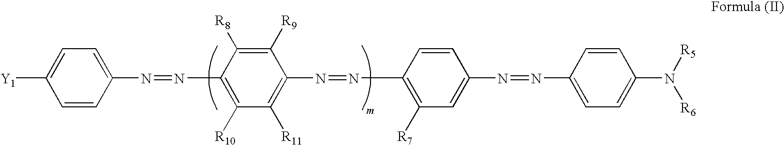
wherein R₅, R₆ and R₇ each independently represent an alkyl group; R₈, R₉, R₁₀ and R₁₁ each independently represent a hydrogen atom or a substituent; Y₁ represents a substituted or unsubstituted, alkyl group, alkenyl group, alkynyl group, aryl group, alkoxy group, alkoxycarbonyl group, acyloxy group, acylamino group, alkoxycarbonylamino group, sulfonylamino group, sulfamoyl group, carbamoyl group, alkylthio group, sulfonyl group or ureido group; and m represents an integer of 1 to 3.

4. The dichroic dye composition according to claim 1, further comprising at least one kind of fluoro-aliphatic group-containing compound represented by formula (III), and/or a polymer comprising at least one kind of polymerization unit of a fluoro-aliphatic group-containing monomer represented by formula (IV) or (V) and at least one kind of polymerization unit of an amide group-containing monomer represented by formula (VI): 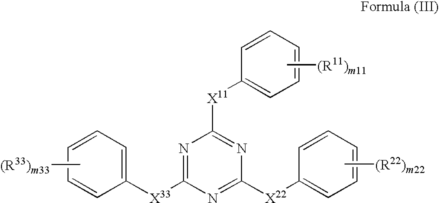
wherein R¹¹, R²² and R³³ each independently represent an alkoxy group having CF₃ group(s) or CF₂H group(s) at its terminal; X¹¹, X²² and X³³ each independently represent —NH—, —O— or —S—; and m11, m22 and m33 each independently represent an integer of 1 to 3; 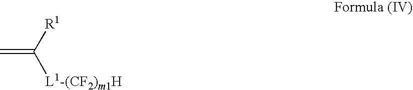
wherein R¹ represents a hydrogen atom, a halogen atom or a methyl group; L¹ represents a divalent linking group; and m1 represents an integer of 1 to 18; 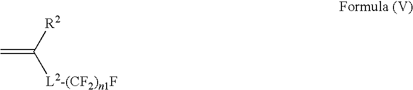
wherein R² represents a hydrogen atom, a halogen atom or a methyl group; L² represents a divalent linking group; and n1 represents an integer of 1 to 18; and 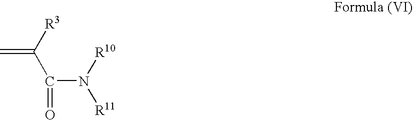
wherein R³ represents a hydrogen atom, a halogen atom or a methyl group; R¹⁰ and R¹¹ each independently represent a hydrogen atom, an alkyl group having 1 to 18 carbon atoms, an aromatic group having 6 to 20 carbon atoms, or a heterocyclic group having 1 to 20 carbon atoms; and R¹⁰ and R¹¹ may be bonded with each other to form a heterocyclic group.

5. A light absorption anisotropic film formed by utilizing the dichroic dye composition according to claim 1.

6. The light absorption anisotropic film according to claim 5, wherein dichroic dye in the light absorption anisotropic film is orientated with the tilt angle at the side of an alignment film of from 0° to 5°.

7. A polarizing element comprising an alignment film and the light absorption anisotropic film according to claim 5 on a support.

8. The polarizing element according to claim 7, wherein dichroic dye in the light absorption anisotropic film is orientated with the tilt angle at the side of the alignment film of from 0° to 5°.

9. A liquid crystal display device comprising the light absorption anisotropic film according to claim 5.

10. A liquid crystal display device comprising the polarizing element according to claim 7.

11. A method of producing the polarizing element according to claim 7, comprising the steps of:
(1) rubbing a support or an alignment film formed on a support;
(2) applying the dichroic dye composition according to claim 1 dissolved in an organic solvent on the rubbing treated support or alignment film; and
(3) orientating the dichroic dye composition by causing the organic solvent to evaporate.
